(19) United States
(12) Patent Application Publication
(10) Pub. No.: US 2026/0283449 A1
(43) Pub. Date: Sep. 24, 2026

(54) Endoscope, base module and handling module for an endoscope, sterile single-use product, system, and method for providing an endoscope

(71) Applicant: KARL STORZ SE & Co. KG, Tuttlingen (DE)

(72) Inventor: Peter Schwarz, Tuttlingen (DE)

(73) Assignee: KARL STORZ SE & Co. KG, Tuttlingen (DE)

(21) Appl. No.: 19/153,840

(22) PCT Filed: Feb. 7, 2024
(86) PCT No.: PCT/EP2024/052978

§ 371 Date: Aug. 5, 2025

(30) Foreign Application Priority Data
Feb. 9, 2023 (DE) 10 2023 103 175.9

(51) Int. Cl. A61B 1/00; A61B 1/05

(52) U.S. Cl. CPC A61B 1/00105; A61B 1/00006; A61B 1/00066; A61B 1/00103; A61B 1/00124; A61B 1/00128; A61B 1/00144; A61B 1/05

(57) ABSTRACT

The present invention relates to an endoscope (20, 220, 320). The endoscope (20, 220, 320) comprises a base module (22, 622). The base module (22, 622) comprises a housing (24, 624) and control electronics (26, 626), wherein the housing (24, 624) encloses the control electronics (26, 626). Further, the endoscope (20, 220, 320) comprises a handling module (28, 628, 828, 928, 1028) configured to make the base module (22, 622) handleable and comprising a main body (30, 630, 830, 930, 1030) that defines an accommodation space (32, 632, 832, 932, 1032) for the base module (22, 622). To establish a handleable state, the base module (22, 622) is introducible into the accommodation space (32, 632, 832, 932, 1032) of the handling module (28, 628, 828, 928, 1028) by a user. Further, the handling module (28, 628, 828, 928, 1028) and the base module (22, 622) together form a securing mechanism (34, 634, 834, 934, 1034) by means of which the housing (24, 624) is releasably securable to the handling module (28, 628, 828, 928, 1028).

The present invention relates to an endoscope, to a base module for an endoscope, to a handling module for an endoscope, to a sterile single-use product, to a system, and to a method for providing an endoscope.

An endoscope is often used to perform a diagnostic and/or therapeutic action on a part of a patient's body that is difficult to access. Parts of the body that are difficult to access are, in particular, cavities such as a body cavity and/or a hollow organ. The endoscope is designed in such a way that it can be partially fed into the patient's cavity in order to perform the diagnostic and/or therapeutic action. In order, for example, to make anatomical structures visible or to better monitor the diagnostic and/or therapeutic action, the endoscope is usually configured to shine light into the patient's cavity.

Endoscopes comprise an elongated shaft that is partially fed into the patient's cavity. To examine an anatomical structure, a distal end of the shaft is aligned so that it points significantly in the direction of the anatomical structure. This allows the anatomical structure to be illuminated in a targeted manner. The light reflected from the anatomical structure can then be captured by the distal end of the shaft for imaging. After imaging, the anatomical structure can be displayed to a user.

For handling, endoscopes comprise a handle and/or a handling section that has an ergonomic shape for better handling. The handle is often milled from a solid metal material, which is sometimes a complex and/or cost-intensive manufacturing step. The handle manufactured in this way is permanently connected to the other parts of the endoscope. In addition, electronic components, operating elements and/or the like may be provided in the handle. However, for some applications of the endoscope, it may be that a different shape of the handle would be advantageous. As a result, the user is faced with the choice of continuing to use the endoscope with the less advantageously shaped handle or using a different endoscope that comprises an advantageously shaped handle.

Furthermore, when using endoscopes for medical purposes, it is important to ensure that they are sterile and can be sterilized as easily and reliably as possible. Consequently, the handle and the entire endoscope in general must meet special requirements regarding tightness, in particular fluid-tightness and/or gas-tightness. The endoscope and in particular the handle must enclose electronics contained therein in such a way that the entire endoscope, including the permanently secured handle, can be sterilized as easily and reliably as possible. This may in particular include autoclaving, which entails a high level of requirements for temperature resistance and gas-tightness.

Furthermore, it may be provided in this regard that the endoscope is partially provided with a sterile disposable cover, a so-called “drape,” in order to at least largely protect critical sections of the endoscope, in particular the handle, from contamination during an intervention. The process of covering the endoscope with the sterile disposable cover can be time-consuming and/or labor-intensive. In addition, the sterile disposable cover is usually intended for single use.

Another aspect related to endoscopes is the wide range of different types of endoscopes, each of which is intended for a specific use. The endoscopes comprise electronic components which are adapted for the respective application, and which can be provided integrally with a handle. This can result in a highly fragmented development process and/or manufacturing processes in which only minor synergies between different types of endoscopes are utilized. For endoscope manufacturers, offering a wide range of products can therefore be associated with comparatively great effort.

Yet another aspect can concern heat build-up during the operation of endoscopes. In particular, waste heat generated by electronic consumers, in particular by luminous elements such as LEDs, can heat the handle, which is made of metal and is permanently connected to the rest of the endoscope. As a result, the handle can heat up to such an extent that the user can hardly grip it and/or the power of the electronic consumers must be reduced. In general, the power of the electronic consumers can be restricted or limited by the heating of the handle. This sometimes results in limitations regarding maximum light intensity, which is not caused by the installed or generally installable luminous elements, but by the undesirable development of waste heat, which can affect the comfort of the user or the handleability of the endoscope.

In addition, one aspect relates to a cable that connects the endoscope to a supply unit. This cable may have a preferred rotational position with respect to the endoscope and/or define a preferred rotational position of the endoscope, which can result in disadvantages when using the endoscope. Connecting the endoscope can therefore require a high degree of attention in order to properly align the components involved and to establish the connection correctly. Furthermore, the cable may be designed in such a way that a cable replacement can only be carried out by trained specialist personnel.

Based on the prior art, it is an object of the present invention to improve the flexibility, efficiency and/or cost-effectiveness of an endoscope.

The object can be achieved according to the invention by an endoscope, a base module for an endoscope, a handling module for an endoscope, a sterile single-use product, a system and a method for providing an endoscope, as described herein and defined in the claims.

According to the invention, an endoscope may be provided. The endoscope may comprise a base module. The base module comprises a housing and control electronics, wherein the housing encloses the control electronics. Further, the endoscope may comprise a handling module configured to make the base module handleable and comprising a main body that defines an accommodation space for the base module. To establish a handleable state, the base module can be introducible into the accommodation space of the handling module by a user. In addition, the handling module and the base module can together form a securing mechanism by means of which the housing is releasably securable to the handling module.

According to the invention, a base module for an endoscope may also be provided.

According to the invention, a handling module for an endoscope may also be provided.

According to the invention, a system may also be provided. The system may comprise a base module and at least two different handling modules, wherein the two handling modules implement different functionalities in a handleable state of the base module. In other words, a first handling module can implement a first functionality in a first handleable state of the base module in which the base module is introduced into the first handling module, and a second handling module can implement a second functionality that is different from the first functionality in a second handleable state of the base module in which the base module is introduced into the second handling module.

According to the invention, a method for providing an endoscope for a user may also be provided. The method may comprise providing the base module, providing the handling module, and introducing the base module into the handling module by the user.

Advantageously, an endoscope may be provided which is characterized by high flexibility, high efficiency and/or low operating costs. The endoscope may be provided so that it can be customized by the user, and consequently the quality of an application of the endoscope can be improved. In addition, development and/or manufacturing costs can be saved. In addition, it is possible to react more quickly and/or precisely to events during use of the endoscope, for example, if it turns out during use that a different handling module is advantageous. The user can optimize the endoscope and in particular the handling according to his own needs and/or requirements. The design of the endoscope can be simplified by providing modules, each of which is assigned a separate function. For example, the handling module can be compatible with various base modules, thus allowing standardized handling modules to be provided. In addition, the overall use of materials and/or energy required for use over a longer period of time can be reduced.

The inventors have found that conventional draping is a comparatively complicated process that regularly requires at least two people. There is a risk of contamination. The invention makes it possible to provide all components in a sterile manner. These can be assembled by a single person. This increases patient safety, in particular with regard to hygiene. In addition, the inventors have found that the costs of a drape are at the same level as the costs of a single-use handling module, in particular if it is designed as an injection-molded part, for example. In both cases, single-use products are disposed of, so there are no significant differences in terms of waste generated. Handling modules according to the invention may even be advantageous in this regard because they are easier to recycle.

The features described herein are generally applicable to an endoscope and/or an endoscope device. The disclosure thus relates in some aspects generally to an endoscope device. The endoscope device may comprise a base module. The base module comprises a housing and control electronics, wherein the housing encloses the control electronics. The endoscope device may comprise a handling module. To establish a handleable state, the base module can be introduced into the accommodation space of the handling module. This can be done during manufacture of the endoscope. This can be done alternatively or additionally by a user. The handling module and the base module can together form a securing mechanism by means of which the housing is securable to the handling module. The securing can be releasable. It is understood that the aspects described below may be combined with the general aspects mentioned.

The disclosure further relates to an endoscope having such an endoscope device. Furthermore, the disclosure relates to a method for producing such an endoscope device and/or such an endoscope.

The endoscope may be a flexible and/or a rigid endoscope. For example, the endoscope may comprise a section that is movable and/or controllable. The base module may be a universally applicable and/or standardized module. The base module may, in itself, implement and/or control and/or combine at least substantially all of the functions of an endoscope with regard to the generation of image data. Taken on its own, the base module can form a complete unit. In particular, the base module may be configured to be sterilizable and/or autoclavable. In particular, the housing may be designed to be fluid-tight and/or gas-tight. The base module may comprise all of the components that are configured to generate image data. For example, the components may comprise at least one image sensor, at least one luminous element, at least one optical system, in particular for imaging, at least one light-guiding device, in particular comprising a rod lens and/or an optical waveguide, at least one electrical conductor and/or at least one actuator, in particular for actuating a section of the base module and/or the optical system, in particular for focusing. Furthermore, at least one of the components, in particular the luminous element and/or the image sensor, can generate waste heat during operation. The base module may be configured to dissipate the waste heat, in particular to a section designed as a heat sink. In this context, the base module may comprise a waste heat dissipation device. In particular, the base module may comprise a heat pipe.

The base module may be compatible with different or differently designed handling modules. It may be provided that the system comprises multiple handling modules which have different features, for example size, scope and/or shape, and/or functionalities, wherein all handling modules are compatible with the same base module, or the base module is releasably securable to all handling modules.

The base module may have a main extension direction. The base module may extend, for example, 5 cm to 200 cm, in particular 10 cm to 150 cm, preferably 20 cm to 100 cm, particularly preferably 40 cm to 80 cm, along the main extension direction. A main extension axis may extend along the main extension direction. A “main extension direction” of a component is to be understood in particular as a direction which runs parallel to a longest edge of a smallest imaginary cuboid which only just completely encloses the component.

The user may be an end user and/or an operator. In particular, the user is not involved in the manufacture of the base module and/or the handling module. In addition, the user is in particular not involved in the preparation of the endoscope in a saleable condition and/or in the production and/or manufacturing of the endoscope prior to its distribution. Rather, the user can use the endoscope in an already purchased condition. The user may be a physician, medical assistant, clinical staff and/or medical personnel. The handleable state is established in particular in a patient-related context. The handleable state is established, for example, immediately before use of the endoscope, for example, at most 60 minutes, at most 30 minutes, at most 10 minutes or even at most 5 minutes before use. The establishment of the handleable state can serve to make the endoscope ready for operation. In particular, the establishment of the handleable state does not take place as part of manufacturing and/or production and/or in a plant and/or factory.

The control electronics comprise electronic components configured to enable imaging using the endoscope. For example, the control electronics may comprise a computing unit such as a microprocessor, a communication and/or network element, a memory element, a voltage converter and/or a motor controller. The control electronics may be configured to supply the elements configured to generate image data, for example the image sensor and/or the luminous element, with electrical energy and/or to control a function. For example, the generation of image data may be capable of being initiated and/or a brightness may be adjustable. Furthermore, the control electronics may be configured to digitize image data, in particular in the case where the image sensor is a CCD sensor and/or a CMOS sensor and/or a silicon image sensor and/or a SWIR image sensor or another digital sensor. The control electronics may generally be configured to operate many different elements. The elements may be provided as required, for example depending on the type of endoscope and/or the area of application of the endoscope, and may be couplable and/or compatible with the control electronics.

In addition, the control electronics may be configured to transmit image data to a supply unit and/or a display unit. The supply unit may be configured to process the image data. The display unit may be configured to display the image data, in particular during operation of the endoscope.

The housing may be configured to absorb waste heat from the control electronics and/or the components of the base module and/or to dissipate it via an outer surface of the housing, in particular to an environment of the housing. The housing may also be configured to enclose the control electronics in a fluid-tight manner.

The handling module may generally provide an interface for mechanical manipulation of the endoscope. “Making handleable” can, for example, mean that the handling module is grippable by a user and/or that the endoscope is movable in space. In addition, the endoscope may be attachable to a robot, in particular a medical robot, by means of the handling module and/or may be grippable and/or holdable by the robot. In this context, the user can establish the handleable state by initiating a particularly automatic establishment of the handleable state by a robot, in particular by operating an interface with the robot. The robot may be configured to perform an action, in particular a diagnostic action and/or an intervention. Furthermore, the endoscope may be attachable to a holding arm using the handling module. The holding arm may be configured to hold the endoscope, in particular to assist the user in performing the action. Furthermore, the handling module may have an ergonomic shape for use by the user. In particular, this can mean that the handling module has a preferred orientation with respect to a user's grip. The handling module may be free of electrical and/or electronic components. The handling module may be free of an electrical energy supply and/or a power supply unit and/or an electrical energy storage device.

The handling module may be compatible with multiple base modules. For example, the system may comprise different base modules, each implementing a different functionality. The different base modules, in particular the housings of the base modules, may be introducible into the accommodation space of the handling module and/or the base modules may be releasably securable to the handling module.

In particular, the main body may form the ergonomic shape on a lateral circumferential surface. The accommodation space may have a size and/or shape that ensures that the base module, in particular the housing, may be introducible into the accommodation space with significantly precise fit. In some embodiments, the base module, in particular the housing, is spaced at least in sections from inner surfaces of the accommodation space and/or from inner walls of the main body in an introduced state of the handling module. “Introducible” can in particular mean capable of being slid in, placed in, plugged in, pushed and/or pressed in.

The securing mechanism may in particular be configured to at least substantially restrict axial mobility of the base module relative to the handling module. Furthermore, lateral and/or rotational relative movement can be restricted. The securing mechanism may comprise at least one movable part. The movable part may be provided on the handling module. Alternatively or additionally, the movable part may be provided on the base module. “Releasably securable” can in particular mean that the handling module can be quickly, easily and/or reversibly secured to the base module. In particular, the handling module can be repeatedly securable to and releasable from the base module without damage. In particular, the base module can be used repeatedly. Damage-free may refer in particular to the base module. In some embodiments, it may be provided that the handling module is designed to be releasably securable in such a way that it is destroyed and/or damaged if it is released. For example, the handling module may be connectable and/or releasable within 30 seconds, in particular within 20 seconds and preferably within 10 seconds. Preferably, “easily” can mean that the handling module is connectable and/or releasable by only a single user, preferably without tools. The securing mechanism can be passive or active. “Passive” can mean that the base module can be activated by introducing the base module into the accommodation space. “Active” can mean that the securing mechanism can be operated and/or activated by the user, in particular when the base module is introduced into the accommodation space. In some embodiments, the securing mechanism may be provided on a section of the base module and/or the handling module that is proximal to the user. Furthermore, it may be provided that the securing mechanism is provided on a proximal end surface of the base module and/or the handling module. Alternatively or additionally, the securing mechanism may be provided on a lateral side surface of the base module and/or the handling module.

In particular, the handling module may be intended for single use. This can mean that the user places the base module, in particular the sterilized base module, into the accommodation space of the handling module, in particular briefly, for example a few minutes, before an action. Once an action has been performed, in particular a therapeutic and/or diagnostic action, the user can release the handling module from the base module. For example, the user can introduce the base module into another handling module. The other handling module may have other application-specific features that are advantageous for a particular application.

According to the invention, a sterile single-use product may also be provided. The sterile single-use product may comprise a handling module and a sterile outer packaging, wherein the handling module is packaged in a sterile state in the outer packaging. Advantageously, the user can quickly make the endoscope handleable. The endoscope may be provided, or the base module may be introducible, in such a way that the endoscope does not have to be sterilized before use. It can enable significant time savings, high levels of convenience and/or flexible and/or rapid response to events during use.

Furthermore, the handling module may be configured to make the base module handleable and operable by the user, and an operable state may be establishable by introducing the base module into the accommodation space. In the operable state, the endoscope can be at least substantially fully usable by the user. Advantageously, the user can, for example, handle an application-specific base module using a handling module that is advantageous for him. In particular, the base module cannot be fully handleable without the handling module. The usability of the endoscope can be achieved by the user connecting the base module to the handling module. The endoscope may be intended to be purchased and/or kept and/or stored disassembled into the individual components of base module and handling module and to be only made ready for operation by assembling these individual components before use.

Furthermore, the base module is releasable from and/or securable to the handling module without tools. Usability may be simple. “Without tools” can in particular mean that the user does not need any additional objects to secure and/or release the base module to/from the handling module.

Furthermore, the securing mechanism may comprise a pretensioned and/or pretensionable pretensioning element. For example, the securing mechanism may comprise a spring, in particular a spiral spring, a torsion spring, a disc spring and/or an annular spring. The pretensioning element can generate a pretensioning force. A securing force may be associated with the pretensioning force. The pretensioning element can be pretensioned by introducing the base module. For example, the base module and/or the handling module may comprise a clamping element, for example a projection, a stop, a ramp, a wedge, an elevation and/or the like, along which the pretensioning element can be guided. The clamping element may in particular extend in a direction in which the pretensioning force acts partially and/or substantially. In addition, the pretensioning force may be provided in a base module-specific manner. For example, in connection with a larger and/or heavier base module, a greater pretensioning force and/or securing force may be provided. In some embodiments, the pretensioning force may act partially and/or at least substantially in a radial direction. The securing mechanism may be characterized by its simple usability. In particular, a single user can secure the base module and/or the base module can be securable by a single user. A high level of operational reliability and/or user-friendliness may be achieved.

In addition, the securing mechanism may comprise a holding element which is configured to releasably secure the housing to the handling module by engaging behind it. The holding element may be provided on the handling module. Alternatively, the holding element may be provided on the base module, in particular the housing. The securing mechanism may comprise further holding elements which are configured to releasably secure the housing to the handling module by engaging behind it. The holding elements may be provided on the handling module and/or the base element, in particular the housing. By engaging behind, a high degree of securing force may be achieved. This may significantly increase operational reliability. In addition, the releasability of the base module may be achieved in a structurally simple manner.

Furthermore, the securing mechanism may comprise at least one rotatable securing element, wherein the rotatable securing element is movable by rotation between a securing state and a release state. In particular, “rotatable” can mean rotatable about a rotation axis. For example, the rotation axis may be arranged coaxially with the main extension direction and/or with a longitudinal axis of the base module. In the release state, the base module can be introducible into the accommodation space of the handling module by a user to establish the handleable state. In some embodiments, an axial force may be exerted on the base element and/or the handling module by the rotatable securing element. The securing mechanism may further comprise a pretensioning element. Such a securing mechanism may be characterized by high reliability and low susceptibility to errors. The user can receive almost immediate feedback about a successful securing. In particular, the securing state can be easily visible to the user.

Furthermore, the accommodation space may be openable and closable. “Openable” can mean that the accommodation space can be made laterally accessible. For example, a lateral side of the accommodation space can be removable. In particular, the accommodation space can also be openable and/or closable by the user. Advantageously, operational reliability and user-friendliness can be increased. The accommodation space can have an open and closed state. In the open state, the base module may be introducible into the accommodation space and/or removable from the accommodation space. In the closed state, the base module may not be introducible into the accommodation space and/or removable from the accommodation space. Opening and/or closing the accommodation space may comprise a mechanical movement of at least one component of the handling module, in particular by the user.

Furthermore, in an open state of the accommodation space, the base module is introducible into the accommodation space and in a closed state of the accommodation space, the housing is securable to the handling module. In particular, the housing may be introducible into the accommodation space. The housing can be introducible and/or securable by a user. In this context, “introducible” can in particular mean “can be placed into.” In particular, the base module, in particular the housing, may be introducible into the accommodation space from a lateral side and/or in a direction perpendicular to the main extension direction and/or longitudinal axis of the base module and/or the handling module and/or the accommodation space. Advantageously, in the closed state, at least the housing is enclosed by the accommodation space, or the accommodation space at least substantially encloses the housing. In particular, the control electronics can be protected in this way. A malfunction can be preventable. Advantageously, operational reliability can be increased. Furthermore, an advantage may be that the base module can be in particular introducible into the accommodation space in such a way that the user grips the base module in the area of the housing. For example, the user can grip the base module by the housing and introduce it into the accommodation space, in particular by placing it in. This may be particularly advantageous if the base module is designed to be more stable and/or robust in the area of the housing relative to other sections of the base module. For example, it may be possible to introduce the base module into the accommodation space without gripping an endoscope shaft. The risk of damage to the base module during the introduction can be significantly reduced.

Furthermore, the main body may comprise at least two main body parts which are connected to one another in an articulated manner and are movable relative to one another for opening and closing the accommodation space. In particular, the two main body parts may be pivotally movable relative to each other and/or define a pivot axis. For example, the pivot axis may be arranged parallel to the main extension direction and/or the longitudinal axis of the base module. This can make the introduction particularly easy and/or safe to operate. Furthermore, the endoscope may be particularly user-friendly.

Furthermore, the main body may comprise a handle. Advantageously, the handle may be formed integrally and/or in one piece with the main body. The handle may be designed in such a way that the user is provided with a high level of grip comfort. In particular, the handle may be irregular and/or asymmetrical. Operating costs and/or manufacturing costs can be reduced.

Furthermore, in the handleable state, the handle can encompass the housing at least in sections. This enables a compact design to be achieved. In addition, the endoscope can be operated and/or handled in a similar way to conventional endoscopes. For example, the handle may fully encompass at least a section of the housing relative to a longitudinal axis of the handle and/or relative to a longitudinal axis of the housing. The handle may define a handle interior completely surrounded on an end face and/or a lateral surface, in which handle interior at least a section of the housing is arranged in an introduced state of the base module. The handle may be cylindrical at least in sections. The housing may be cylindrical at least in sections.

Furthermore, in the handleable state the handle may, at least at least in sections, be arranged distally with respect to the housing. For example, a circumference of the base module may be larger in the area of the housing than distal to the housing. The housing and/or the handle may be designed in such a way that the handle can be at least substantially gripped by a hand of the user, or the handle can be substantially enclosed by the hand. The handle may also be designed in such a way that the handle can be partially gripped by the hand. Distally in relation to the housing, the circumference may be, for example, 0.1, 0.2, 0.3, 0.4, 0.5, 0.6 times the circumference in the area of the housing. In particular, the section of the handle arranged distally with respect to the housing may be pen-shaped. Pen-shaped can mean that the section can be gripped by the user as if it were a pen being gripped for writing. Accuracy can be increased by such a handle. The endoscope can be aligned more easily, precisely and/or accurately by the user, for example with an anatomical structure. The user has a greater sensitivity in positioning an elongated object when gripping it like a pen for writing than when gripping it with one hand and/or, for example, enclosing it in a fist.

Furthermore, in the handleable state, the handle may be arranged, at least in sections, proximally with respect to the housing. Furthermore, in the handleable state, the handle may be arranged at least substantially laterally and/or radially externally with respect to the housing.

Furthermore, a longitudinal axis of the handle may correspond to a longitudinal axis of the accommodation space. For example, the base module may be arranged substantially coaxially with the handle, in particular in the introduced state of the base module. Advantageously, a compact endoscope that is inexpensive to manufacture can be provided in this way.

Furthermore, a longitudinal axis of the handle may be inclined with respect to a longitudinal axis of the accommodation space. The longitudinal axis of the handle and the longitudinal axis of the accommodation space may, for example, enclose an angle of 0° to 179°, in particular of 25° to 145°, preferably of 45° to 120° and particularly preferably of 75°to 100°. Advantageously, the ease of use can be increased. Furthermore, the endoscope can be more ergonomically grippable and/or handleable.

Furthermore, the handle may be a pistol grip. A pistol grip is characterized by particularly high ease of use and an ergonomic handle. “Ergonomic” can mean that the user's wrist does not need to be bent and/or need only be slightly bent while using the endoscope. In particular, the pistol grip may be advantageous if the main extension direction of the base module extends at least substantially perpendicular to the frontal plane of the user during use.

In addition, the handle may have a smaller diameter than the housing. Advantageously, the diameter of the handle may be designed such that the endoscope, in particular the handle, can be easily and comfortably gripped by the user. The handle may have sections with different diameters, wherein at least one section can be present whose diameter is smaller than the diameter of the housing.

Furthermore, the handle may have an ergonomic shape. A high level of comfort can be achieved. In addition, it can be ensured that the endoscope can be handled by the user over a longer period of time. An endoscope that is ergonomic for the user may be provided. The ergonomic shape may be a shape adapted to a human hand. The handle may have shaping elements that are adapted in size, spacing and/or orientation to a human palm and/or to a palmar side of at least one human finger. This information refers in particular to the average anatomy of an adult human, for example a woman and/or a man.

In addition, the handling module and the base module may together form an alignment aid which defines a preferred position and/or a preferred orientation of the housing relative to the handling module. The preferred position and/or the preferred orientation may be defined in particular with respect to a property of the base module. The preferred position and/or the preferred orientation may in particular be a radial preferred position and/or preferred orientation. Alternatively or additionally, the preferred position and/or the preferred orientation may in particular be an axial preferred position and/or preferred orientation. The radial preferred position and/or preferred orientation may in particular relate to the position of the longitudinal axes relative to one another. For example, the base module may be designed asymmetrically. The alignment aid enables repeatable and error-free introduction of the base module.

In addition, the alignment aid may have at least one guide lamella and a guide groove corresponding to the guide lamella. In some embodiments, the guide lamella and/or the guide groove may extend along the longitudinal axis of the housing and/or the handle. In addition, multiple guide lamellae and/or guide grooves may be provided. For example, the alignment aid may comprise two, three, four, five and/or six guide lamellae and/or guide grooves. Guide lamellae and/or guide grooves are characterized by particularly secure and/or reliable guidance. The base module may be guided particularly securely in the handling module. Furthermore, a high level of operational reliability can be achieved.

In addition, the base module may comprise at least one actuating element by means of which at least one function of the base module can be controlled, and the handling module may comprise at least one operating element which, in the handleable state, acts on the actuating element in such a way that an operation of the operating element, in particular by the user, causes an actuation of the actuating element. The base module may comprise one, two, three, four, five or six actuating elements and/or the handling module may comprise one, two, three, four, five or six operating elements. In particular, the base module may comprise more actuating elements than the handling module comprises operating elements. A controllable function of the base module may, for example, comprise an image recording function, in particular a recording start, a recording stop and/or a recording frequency, an illumination function, in particular an illumination light wavelength and/or an illumination light intensity, a base module mode, comprising, for example, a white light mode, a multi-and/or hyperspectral mode, a red light mode, a blue light mode, a green light mode, a video mode, a guidance mode in which an application is supported by guidance elements displayed together with the image data, and/or a focusing mode, a control of an actuator, in particular an actuator which is configured to move at least a section of the base module, and/or a cooling function, in particular of the base section. Action can include non-contact action. The actuating element may, for example, comprise a sensor that at least partially detects a movement of the operating element. For example, it may also be possible to detect a switching position of the operating element at which the action can be triggered. Alternatively or additionally, the operating element may be provided in such a way that it acts mechanically on the actuating element. Multiple switching positions, each of which can be actuated, may be provided for each operating element and/or actuating element. The operating element may comprise a knob, a button, a switch and/or the like. In particular, the operating element may be mounted radially in the handle, in particular in the main body. The handle, in particular the main body, may comprise an operating element guide arrangement. In particular, the operating element guide arrangement may comprise a linear guide. Advantageously, the handle does not need to comprise an aperture to the base module that is designed to allow actuation by the user.

In addition, the operating element may be rotatable and an actuation of the actuating element may take place in accordance with a rotational position of the operating element. Advantageously, a function may be gradually adjustable. In particular, this may be advantageous in connection with adjusting the illumination light intensity, controlling the movable section of the base module, and/or a focal plane of the at least one image sensor.

In addition, the actuating element and/or the operating element may comprise at least one magnet. An actuation and/or an actuation detection and/or an actuation transmission may be based on a magnetic force. In particular, the magnet may be a permanent magnet. Magnets can be inexpensive components that are available in large quantities. The endoscope, in particular the handling module, may be inexpensive to manufacture and/or simple in terms of design.

Furthermore, the actuating element may comprise a Hall sensor. Hall sensors are particularly characterized by the fact that they require only a small installation space, are insensitive to interference, for example from liquids, dust, dirt, high and/or low temperatures and/or low and/or high brightness, and/or may have a high switching speed. Furthermore, Hall sensors may be inexpensive to purchase and/or operate. Advantageously, a reliable and/or error-free endoscope may be provided.

In addition, the base module may comprise at least two functionally identical actuating elements which are arranged at different circumferential positions, in particular opposite one another with respect to a longitudinal axis of the accommodation space, and which are each configured to interact with the operating element. For example, the base module may be rotatable by 180° around the longitudinal axis of the housing and may be introducible into the handling module in the corresponding rotational position. This may be particularly advantageous if the base module is designed asymmetrically at least in sections. Advantageously, a handling module may be provided for operating the base module in at least two rotational positions of the base module, in particular without requiring a rotation of the handling module in this regard. For example, the user can release the base module and remove it from the handling module. The user can then rotate the base module around the longitudinal axis, in particular by 180°, and introduce and/or secure the base module in the new rotational position in the handling module.

The base module may further comprise a shaft having a distal section and having a proximal section, wherein the proximal section is attached to the housing. The distal section is in particular configured to be introduced into and/or located in a cavity to be examined in an operating state, for example during the diagnostic and/or therapeutic activity. The proximal section is in particular configured to be arranged outside the cavity to be examined in an operating state, for example during the diagnostic and/or therapeutic activity. “Distal” should be understood to mean, in particular, facing a patient and/or facing away from a user during use. “Proximal” should be understood to mean, in particular, facing away from a patient and/or facing a user during use. In particular, proximal is the opposite of distal. The shaft may be an elongated object. Furthermore, the shaft may at least partially and preferably, at least to a large extent, form the distal section. For example, the shaft may have a smaller diameter than the housing. The shaft may be configured to be introduced into a body cavity of the patient. The shaft may have a diameter, in particular a main diameter, which significantly defines the shaft, for example from 1 mm to 50 mm, in particular from 4 mm to 25 mm, preferably from 7 mm to 15 mm. The shaft may be arranged coaxially with the longitudinal axis of the housing. Furthermore, the shaft may be rigid and/or flexible. In particular, the distal section may be provided to be flexible, controllable and/or bendable. In particular, the shaft may comprise mechanical elements that make the shaft controllable. The shaft may extend along the main extension direction of the base module. In particular, the shaft may significantly define the main extension direction. The shaft may, for example, comprise 20% to 95%, in particular 40% to 90%, preferably 60% to 85%, of a main extension of the base module along the main extension direction. In addition, the shaft may comprise the heat pipe and/or the cable. In particular, the heat pipe and/or the line may extend along the shaft, in particular from the distal section to the proximal section. The heat pipe and/or the line may protrude beyond the proximal section, particularly into the housing. The line may be connected to the control electronics. In particular, the line may be configured to connect controllable and/or electronic elements to the control electronics.

Furthermore, the shaft may be designed as an interchangeable shaft. The interchangeable shaft may be releasably connected to the base module and/or the handling module. In particular, the endoscope may comprise a connecting unit, in particular a connector, which is configured to connect electronic elements of the interchangeable shaft to the control electronics. In addition, different interchangeable shafts, or interchangeable shafts that implement different functionalities, may be provided. Advantageously, the interchangeable shaft may be releasably connected to the housing without tools.

Furthermore, the distal section may comprise a camera and/or a light source. The camera may comprise the image sensor and/or the optical system. The image sensor may be, for example, a CCD chip or a CMOS chip. According to some embodiments, the camera may comprise more than one image sensor. For example, the camera may be a stereo camera and/or comprise two image sensors. In some embodiments, the camera may be configured to generate digital image data. Alternatively or additionally, the camera may be configured to generate analog image data. In particular, the camera may comprise a computing unit configured to generate digital image data from analog image data. The light source may comprise the luminous element. The light source may further comprise multiple luminous elements. According to some embodiments, the light source may comprise an LED and/or a laser diode. At least one of the luminous elements may be a white light luminous element, in particular a white light LED. The control electronics may be configured to supply the light source with application-specific and/or operating state-specific electrical energy. “Application-specific/operating state-specific” may in particular mean that a light intensity and/or an emission spectrum of the luminous element can be adjusted with respect to an application/operating state. In some cases, this may be achieved by mixing light from different luminous elements and/or selectively activating and/or deactivating certain luminous elements for different applications/operating states. The light intensity can be adjusted automatically and/or by the user. The connecting unit may be configured to connect the camera to the control electronics and to transmit image data from the camera to the control electronics. Advantages may arise from the fact that the distal section comprises the camera and/or the light source. It may be particularly advantageous that light received by the distal section does not have to be guided to a proximal end of the shaft by means of optical elements such as glass fibers, light guides, rod lenses or the like. This can, for example, reduce and/or avoid imaging errors that may occur when the light is guided along the shaft. Furthermore, small shaft diameters may be achieved because the shaft only needs to contain electrical lines, but not optical components. Furthermore, no light guide needs to be provided to conduct illumination light to the distal section. Overall, high image quality can be achieved with a small shaft diameter.

Furthermore, the distal section may comprise at least one optical element defining a main viewing direction, wherein the main viewing direction is inclined with respect to a longitudinal axis of the shaft. Furthermore, the main viewing direction may coincide with the main extension axis and/or the longitudinal axis of the housing. The optical element may, for example, be a lens of the optical system. Furthermore, the optical element may be translucent in such a way that light can be admitted into the distal section from outside the shaft in a virtually unchanged manner, particularly with regard to spectral components and/or an image plane. According to some embodiments, the main viewing direction may enclose an angle with the longitudinal axis of the shaft and/or the main extension axis. The enclosed angle may be the reference dimension for an angle specification of the main viewing direction. This can mean that if the main viewing direction is parallel to the longitudinal axis of the shaft, the main viewing direction is 0°. The main viewing direction may generally be 0° to 90°. Preferred main viewing directions are at least 0°, 10°, 20°, 30°, 40°, 45°, 60°, 75°, 90°. The main viewing direction may be oriented within an angular range of +/−5° starting from one of these values. The image sensor may be arranged orthogonally to the main viewing direction. According to further embodiments, the image sensor may be arranged such that the longitudinal axis of the shaft extends partially along the image sensor. The inclined main viewing direction may expand the area that can be imaged and/or viewed through the endoscope. In particular, the base module may be rotatable by 180° around its longitudinal axis and may be reintroducible into the handling module. This can be particularly advantageous if the main viewing direction is inclined. For example, the user can enlarge the observation space in this way, whereby the handling module is usable in a preferred orientation.

In addition, the endoscope may comprise a supply cable which is connected and/or connectable to the base module. The supply cable may be configured to transmit electrical energy, analog and/or digital data, in particular image data and/or control signals. “Connectable” can mean that the supply cable can be connected easily and/or quickly. In particular, no specialist personnel is required for the connection. Advantageously, there is no need for any link between the handling module and the supply cable. In particular, the supply cable may be connected and/or connectable independently of the handling module.

Furthermore, the supply cable may be permanently connected to the base module. Advantageously, tightness, in particular gas-tightness and/or fluid-tightness, can be ensured. In particular, the supply cable may be configured to be sterilized and/or autoclaved. Incorrect operation can also be prevented.

In addition, the base module and the supply cable may together form a plug connector. Advantageously, the plug connector may be designed independently of the handling module. This allows a simplified design to be achieved and/or differently designed handling modules to be used.

Furthermore, the supply cable may be a coaxial cable. Furthermore, the base module, in particular the control electronics, may comprise a serializer and/or filter which is/are configured to bundle multiple data signals and/or power-related signals and/or currents in a differential pair and/or to provide them for communication and/or an electrical energy supply via a differential pair. Due to the provision of the coaxial cable, there is no preferred plugging orientation of the supply cable. The operation of the endoscope, in particular plugging in the supply cable, may be simplified and quick. In addition, the risk of incorrect operation can be reduced. In particular, plugging in the supply cable does not require any special attention from the user.

In addition, the supply cable may be rotatable relative to the base module in a connected state. “Rotatable” can in particular mean that the supply cable is rotatably mounted. For example, the supply cable, in particular the coaxial cable, may be secured only against axial displacement. Advantageously, there is no preferred orientation of the supply cable with respect to the base module. The base module may be rotated during operation. In particular, the risk of twisting and/or knotting of the supply cable may at least be reduced. This can increase operational reliability.

In addition, a cable outlet direction of the supply cable from the base module may be inclined with respect to a longitudinal axis of the base module. “Cable outlet direction” can mean that the supply cable, in a close range to the base module in a connected state, comprises a rigid section which deflects an extension direction of the supply cable. For example, this can prevent the supply cable from being in the user's way and/or the user from damaging the supply cable. This can improve the operational reliability and/or durability of the endoscope.

Furthermore, the main body may form on its proximal side an opening through which the base module is introducible into the accommodation space. In this context, “introducible” can in particular mean “capable of being slid in.” In particular, the opening may have a diameter that corresponds at least to the diameter of the base module, in particular of the housing. Advantageously, the base module may be grippable on the housing for the introduction. In particular, it may be possible to avoid having to grip the base module by the shaft. Gripping the shaft may, for example, lead to plastic deformation of the shaft and/or damage to components encompassed by the shaft, in particular rod lenses. Furthermore, the introduction can be capable of being carried out quickly and easily by a user. In particular, it may be possible to carry out the introduction during treatment and/or examination of a patient.

Furthermore, the control electronics may be enclosed in a gas-tight manner. In particular, the housing may enclose the control electronics in a gas-tight manner. Advantageously, this makes it possible to provide a sterilizable, easy-to-sterilize and/or autoclavable base module.

Furthermore, the handling module may be configured to be autoclaved and/or sterilized separately from the base module. The handling module may be provided in the sterile outer packaging. The user can remove the handling module from the sterile outer packaging. This makes it possible, for example, to change the handling module quickly and reliably, in particular without the user having to worry about the sterilization status of the handling module. The handling module may be always available and/or quickly ready for use. This can mean, for example, that the user can have various sterile handling modules available during the treatment of the patient. The base module may be introducible into the handling module if required. However, the user does not need multiple base modules, which can reduce the cost of treatment. After treatment, the related handling module and the related base module may be autoclavable and/or sterilizable separately. This enables them to be provided so as to be ready for further treatment. Multiple sterile single-use products may be provided, each of which may comprise a sterile outer packaging and at least one handling module. The sterile single-use product may in particular be storable and/or able to be kept in stock.

In addition, the handling module may be manufactured at least partially and in particular at least largely from plastics material, in particular a polymer material. The phrase “at least largely” should be understood to mean in particular at least 55%, preferably at least 65%, preferably at least 75%, particularly preferably at least 85% and very particularly preferably at least 95%, and advantageously completely, in particular with reference to a volume and/or a mass of a component. The plastics material may be and/or comprise a thermoplastic plastics material and/or a thermosetting plastics material and/or an elastomer. In addition, the main body may be manufactured from sustainable materials, in particular degradable and/or recyclable materials. The plastics material may be and/or comprise a bio-plastics material and/or a bioplastic, for example a plastics material based on renewable raw materials and/or a plastics material that is biodegradable. Advantageously, a cost-effective handling module may be provided. In addition, the plastics material may have a heat-insulating effect, in particular compared to a metal. The handling module can therefore become more handleable by the user. In particular, heating of the handling module, in particular the handle, beyond a limit temperature that the user finds just comfortable can be prevented. This makes it possible to provide, if required, a larger number of luminous elements and/or more powerful luminous elements or to use a higher power when operating luminous elements. In emergency situations in particular, this allows a higher maximum illumination intensity to be used, at least temporarily, without making handling impossible due to the temperature, because the user can no longer hold the endoscope safely due to it heating up.

In addition, the main body may be manufactured by injection molding and/or additively. The main body may be formed integrally and/or in one piece. In particular, the main body may be an injection-molded part. In further embodiments, the main body may be formed in two parts and/or multiple parts. The main body may in particular be permanently assembled, for example if the main body is formed in two parts and/or multiple parts. Furthermore, the main body may be characterized by low-cost manufacture and/or low material usage. Particularly simple producibility can be achieved in particular if the main body can be demolded as a whole, in particular in a direction parallel to the main extension direction and/or parallel to the longitudinal axis of the main body. In some embodiments, the main body may be manufactured from two half-shells which are, for example, bonded and/or welded together, for example by means of ultrasonic welding.

Furthermore, the handling module may be a single-use product. In particular, the handling module can replace and/or eliminate the need for the sterile disposable cover. In particular, providing the sterile disposable cover may be time-consuming. By eliminating the need for the sterile disposable cover, a significant time saving can be achieved. Furthermore, the entire process can be simplified and/or the operation of the endoscope can be more economical. Furthermore, fewer personnel can be required to operate the endoscope.

Furthermore, in the operable state, a longitudinal axis of the base module may coincide with a longitudinal axis of the accommodation space. In particular, the longitudinal axis of the base module may coincide with the main extension axis and/or with the longitudinal axis of the shaft. In particular, the base module may be designed to be rotationally symmetrical, particularly with regard to its shape. The endoscope may have a simple design with little required installation space.

In addition, the handling module may have at least one sealing element which seals against the base module in the operable state. In particular, in the operable state, the accommodation space may not be completely filled by the base module. In other words, there may be a free space between the base module and the walls of the handling module. The at least one sealing element may be configured to seal the accommodation space in a gas-tight and/or fluid-tight manner. For example, the handling module may have the at least one sealing element only on one side, in particular an end face and/or a distal side. The at least one sealing element may seal against the base module, in particular on a distal side of the handling module. In particular, the sealing element may seal against the base module in a close range around the shaft. Advantageously, contamination of an inner side of the handling module and/or the base module, in particular of the housing of the base module, can be prevented and/or at least significantly reduced. Furthermore, it is possible, in particular, to prevent a fluid from flowing along the shaft into the free space between the handling module and the base module, for example. The endoscope can therefore be cheaper and more reliable in operation and can be operated over a longer period of time. In addition, cooling of the base module can be unaffected and/or only slightly affected

The handling module may further comprise at least one air inlet on a distal section of the handling module and at least one air outlet on a proximal section of the handling module, wherein the at least one air inlet is connected to the at least one air outlet. This allows air circulation to be achieved, which may be particularly advantageous for cooling the base module by convection. In particular, air circulation can be achieved in the free space. In particular, it may be achieved that the control electronics can be maintained below a limit temperature range. Above the limit temperature range, the function of the control electronics may be restricted. Furthermore, it may be possible for waste heat to be dissipated, for example, from the distal section of the shaft, in particular by means of the heat pipe along the shaft to the housing. The waste heat can in particular heat up the housing. In this regard, the housing can function as a heat sink. In addition, the base module may comprise a further heat sink, for example a cooling element, which can comprise cooling fins, and/or a copper plate. In addition, the base module may comprise a section which has an enlarged surface, for example by providing cooling fins, and/or is formed from a material which has good heat transfer properties. The air inlet may be configured to admit air, in particular cooling air, into the accommodation space, wherein the air can be used to cool the base module, the control electronics, the housing and/or the further heat sink. Advantageously, control electronics, a camera and/or a light source with higher power may be provided. In addition, the operation of the endoscope may be more comfortable for the user. In particular, heating of the handling module, in particular of the handle, may be prevented and/or reduced.

Furthermore, it may be particularly advantageous to provide the air inlet and/or the air outlet if the handling module is manufactured from a plastics material. Due to the heat-insulating effect of the plastics material, cooling of the base module can be significantly reduced. By providing the air inlet and/or the air outlet, cooling of the base module can be ensured, in particular despite the provision of a handling module made of plastics material. Advantageously, the handling module and the base module can have a synergy through the provision of the cooling effect. The handling module can be inexpensive and/or convenient and the base module can be powerful at the same time.

Furthermore, the at least one air inlet may be connected to the at least one air outlet via an air duct, wherein in the handleable state a radial inner surface of the air duct is defined by the housing. Furthermore, a radial side surface of the air duct may be defined by the alignment aid, in particular by the at least one guide lamella. In addition, a radial outer surface of the air duct may be defined by the handling module. In particular, the air duct may extend along a large part of the housing. For example, the air duct may extend along at least 30%, in particular at least 60% and/or preferably at least 80% of the housing, in particular along the longitudinal axis of the housing. Advantageously, the housing may be cooled particularly efficiently. By directing the air directly along the housing, cooling performance can be high.

Furthermore, the air duct may be hollow cylindrical. This may mean that the air duct extends significantly around the entire circumference of the housing and/or of the base module. In particular, the air duct may surround and/or enclose the housing. The surface area of the base module, in particular of the housing, provided for cooling can be increased and/or particularly efficient cooling can be achieved.

In addition, the handling module may comprise a coolant communication interface via which a cooling fluid is introducible into the accommodation space. It may be possible for the cooling fluid to be directed to the coolant communication interface and/or discharged from the coolant communication interface, in particular by means of tubes. In particular, the system may comprise a cooling fluid conveying device. The cooling fluid conveying device may comprise a pump and/or a heat exchanger. For example, the cooling fluid may be conveyed to the handling module in a first state by means of the pump. In the accommodation space, the coolant may in particular come into contact with the base module, or absorb enthalpy from the base module, in particular to cool the base module. By absorbing the enthalpy, the cooling fluid may enter a second state. Furthermore, it may be possible for the cooling fluid to be conveyed to the cooling fluid conveying device, in particular to the heat exchanger. The heat exchanger may be configured to transfer the cooling fluid from the second state to the first state. The cooling fluid may be a special cooling fluid with application-specific and/or operation-specific properties. In addition, the cooling fluid may be water, in particular distilled water. Cooling by means of cooling fluid may in particular achieve better cooling performance than cooling by means of air and/or convection.

Furthermore, in the handleable state, the accommodation space may be sealed off from the environment. In particular, this may achieve a better and/or more efficient cooling effect. For example, cooling air may be introducible into the accommodation space via an air inlet duct. For example, the air may be introducible at a pressure that is greater than the ambient pressure. Air circulation may be achieved by means of the sealing such that air can be forced to escape from at least one air outlet. As a result, the air circulation can be more efficient and/or adjustable.

Furthermore, the endoscope may comprise a cooling module, wherein the main body defines a cooling module accommodation space into which the cooling module is introducible, and wherein in an introduced state of the cooling module in the handleable state, the cooling module and the base module are at least thermally in contact. Advantageously, the cooling module may be provided for cooling the base module. If the cooling performance of the cooling module decreases during operation of the endoscope, the cooling module can be replaced by an additional cooling module that has the same properties as the cooling module. In particular, the user can replace the cooling module with the additional cooling module. The base module may then be efficiently cooled again using the additional cooling module. In particular, the cooling module may be designed in such a way that it can absorb and/or store a large amount of enthalpy in a small installation space. Advantageously, good cooling performance can be achieved.

The cooling module may also comprise a latent heat accumulator and/or be designed as such. The latent heat accumulator may be configured to store absorbed thermal energy, in particular heat, in the form of latent heat. For example, the absorbed energy may be stored by a phase change, for example from solid to liquid, of a medium. The latent heat accumulator may comprise a phase change material. Advantageously, good heat storage properties can be achieved in a small installation space.

In addition, the cooling module accommodation space may open into the accommodation space, and in the handleable state the cooling module can be brought into physical and thermal contact with the base module by being introduced into the cooling module accommodation space. This allows thermal energy to be transferred particularly efficiently. The cooling module can be pressed against the base module during introduction, allowing both physical and thermal contact to be established. The cooling module can rest against the side of the base module. The design may also be simpler, in particular because no further heat-conducting elements are provided. The endoscope may be more cost-effective and efficient.

In addition, the base module and/or the cooling module may have a heat-conducting deformable element which, in the handleable state and in the introduced state, rests against the cooling module or the base module. For example, if two rigid bodies are brought into contact with each other for heat exchange, heat-insulating air gaps can form between the two bodies. This can reduce and/or impair the heat transfer. By providing the heat-conducting element, the air gaps can be fillable, for example. This can mean that the surface area significantly involved in heat transfer is increased. In principle, a heat exchange surface area can be increased. Consequently, the efficiency of the endoscope and/or a cooling effect can be increased. In particular, a more powerful base module may be provided. In addition, comfort for the user can be improved. The heat-conducting deformable element may, for example, be made of a heat-conducting rubber. Alternatively or additionally, a deformable metallic structure may be provided.

In addition, in the handleable state and in the introduced state of the cooling module, the cooling module can apply a force to the base module. In particular, an interaction with the deformable element can be achieved. For example, the deformable element may be subjected to a force in such a way that the heat exchange surface area is increased and/or thermal contact is improved. Furthermore, the heat exchange surface area may generally be increased. Consequently, the efficiency of the endoscope and/or a cooling effect can be increased. In particular, a more powerful base module may be provided.

Furthermore, the handling module may be configured to be attached to a holder of a medical robot and to make the base module handleable by the robot. In addition, the handling module and the holder may together form a component. Furthermore, the handling module may be permanently attached and/or attachable to the robot. Advantageously, the base module may be made quickly and efficiently handleable by the robot. In particular, the base module may be quickly and efficiently replaced by another, in particular differently designed, base module.

The devices and systems according to the invention and the methods according to the invention should not be limited to the application and embodiment described above. In particular, they may have a number of individual elements, components and units as well as method steps, which differ from a number mentioned herein, in order to fulfill a function described herein. In addition, for the ranges of values specified in this disclosure, values within the stated limits shall also be deemed to be disclosed and to be usable in any manner.

It is in particular pointed out that all features and properties described with regard to a device, but also procedures, can be analogously transferred to methods and can be used within the meaning of the invention and are considered to be co-disclosed. The same applies in the opposite direction. This means that structural features mentioned in relation to methods, i.e., features relating to the device, may also be taken into account, claimed and also counted as part of the disclosure within the scope of the device claims.

The invention is explained below using figures by way of example. The drawings, the description, and the claims contain numerous features in combination. A person skilled in the art will also, expediently, consider the features individually and use them in combination as appropriate in the context of the claims.

If there is more than one example of a particular object, only one of them may be provided with a reference sign in the figures and in the description. The description of this example may be transferred accordingly to the other examples of the object. If objects are named using number words, such as first, second, third object, etc., these are used to name and/or assign objects. Accordingly, for example, a first object and a third object may be included but not a second object. However, a number and/or sequence of objects could also be derived using numerical words.

In the drawings:

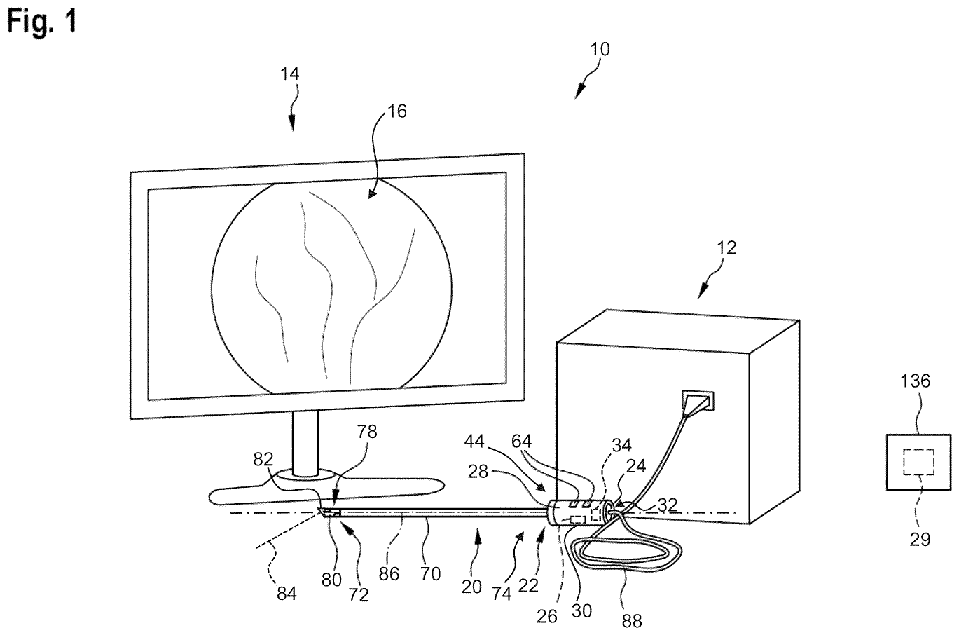

FIG. 1 shows a schematic representation of a system with an endoscope;

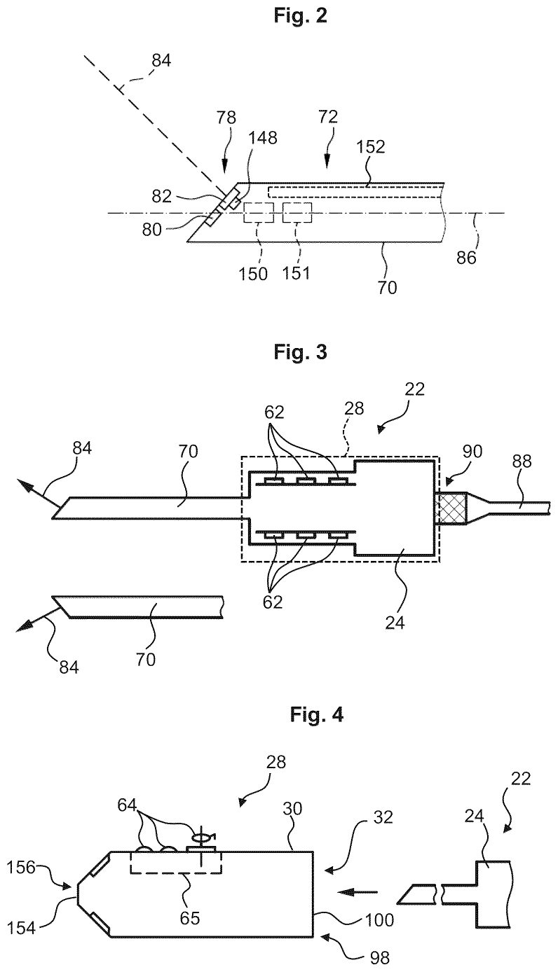

FIG. 2 shows a schematic representation of a distal end of a shaft of the endoscope;

FIG. 3 shows a schematic side view of a base module of the endoscope;

FIG. 4 shows a schematic side view of a handling module of the endoscope;

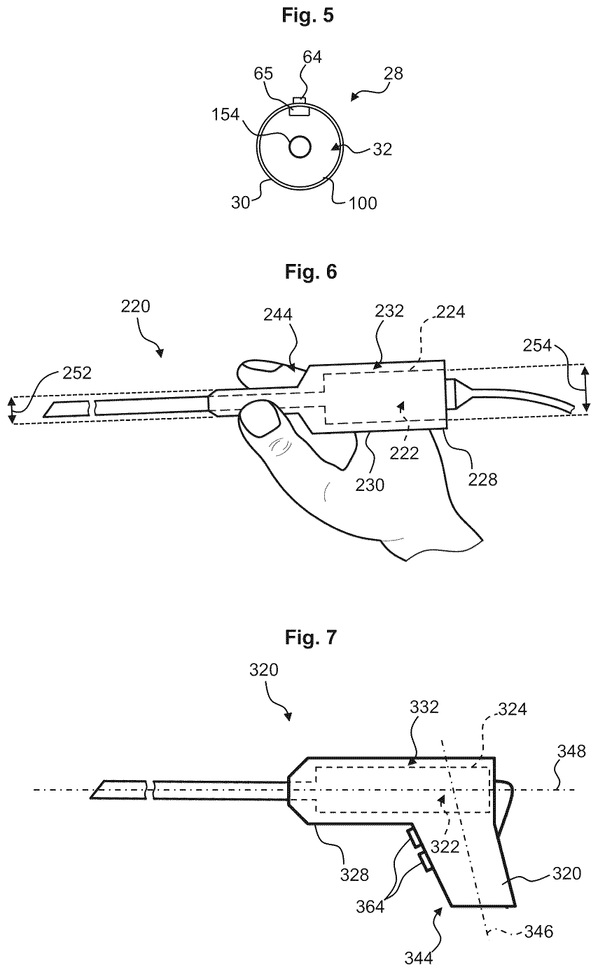

FIG. 5 shows a schematic representation of the handling module viewed from a proximal side of the handling module;

FIG. 6 shows a schematic representation of a further endoscope;

FIG. 7 shows a schematic representation of yet another endoscope;

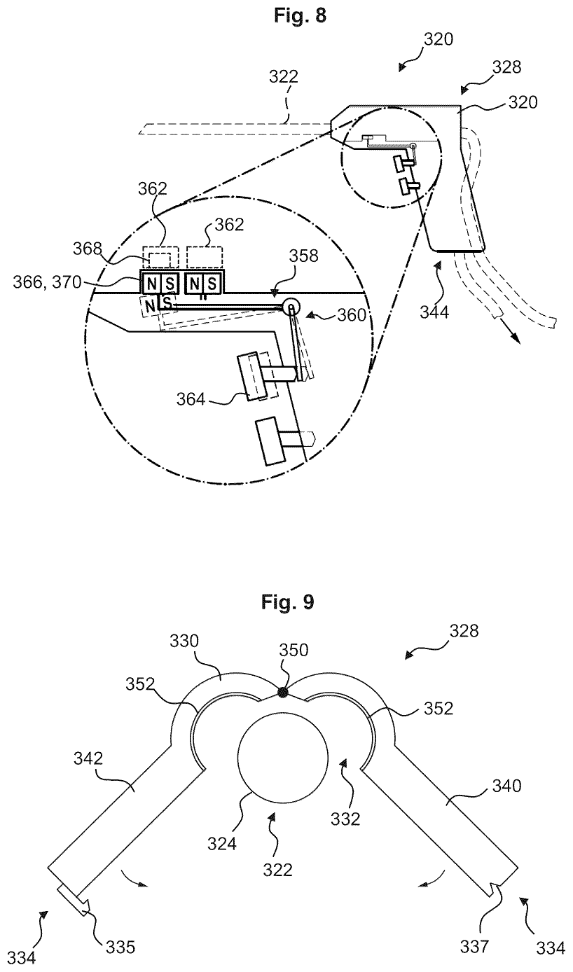

FIG. 8 shows a further schematic representation of the endoscope from FIG. 7;

FIG. 9 shows a schematic representation of a main body of a handling module of the endoscope from FIG. 7;

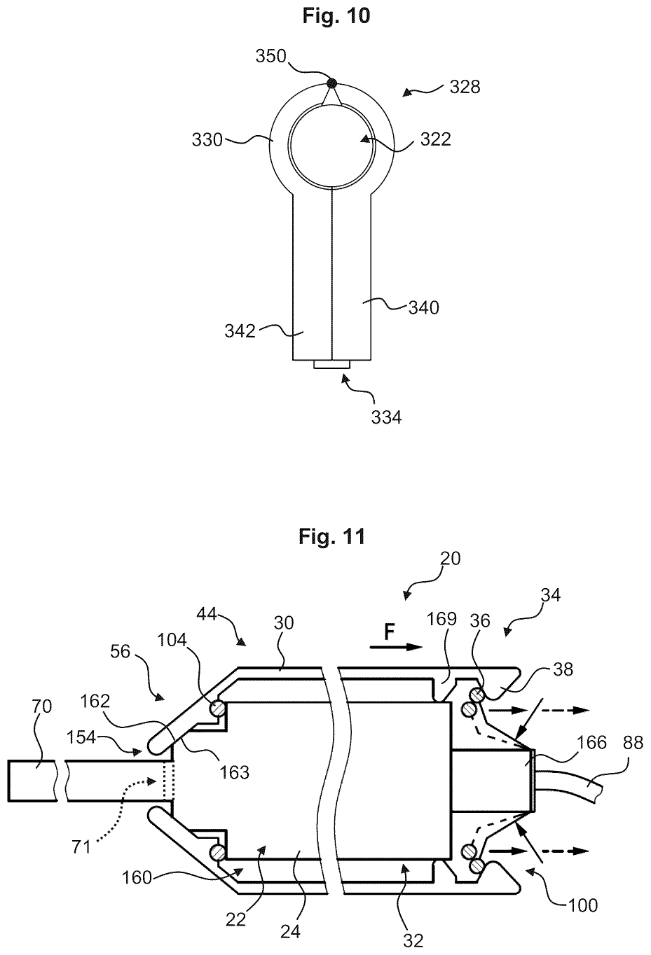

FIG. 10 shows a schematic representation of a securing mechanism;

FIG. 11 shows a schematic representation of the securing mechanism in a view of its proximal side;

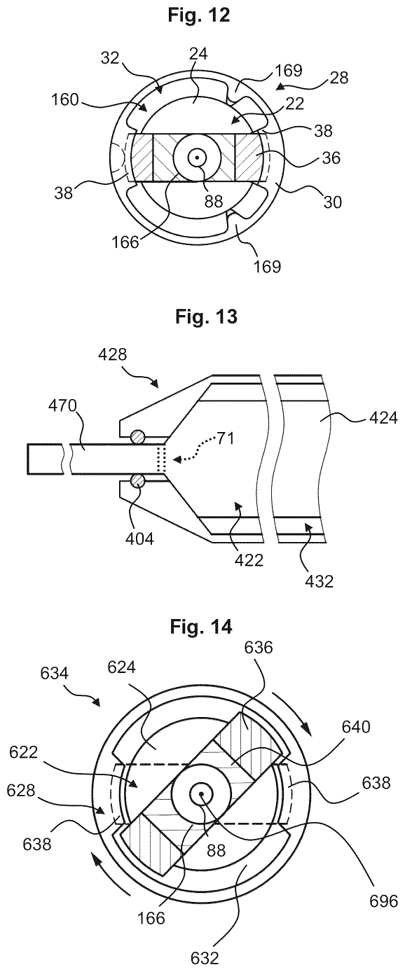

FIG. 12 shows a schematic representation of a further handling module;

FIG. 13 shows a schematic representation of a further handling module and a further base module;

FIG. 14 shows a schematic representation of a further securing mechanism;

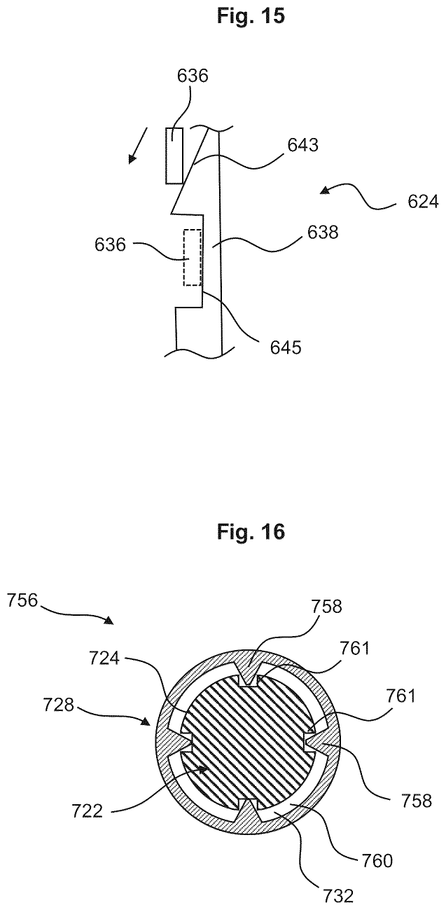

FIG. 15 shows a schematic representation of a holding element;

FIG. 16 shows a schematic representation of a view of a transverse section through a further base module and a further handling module;

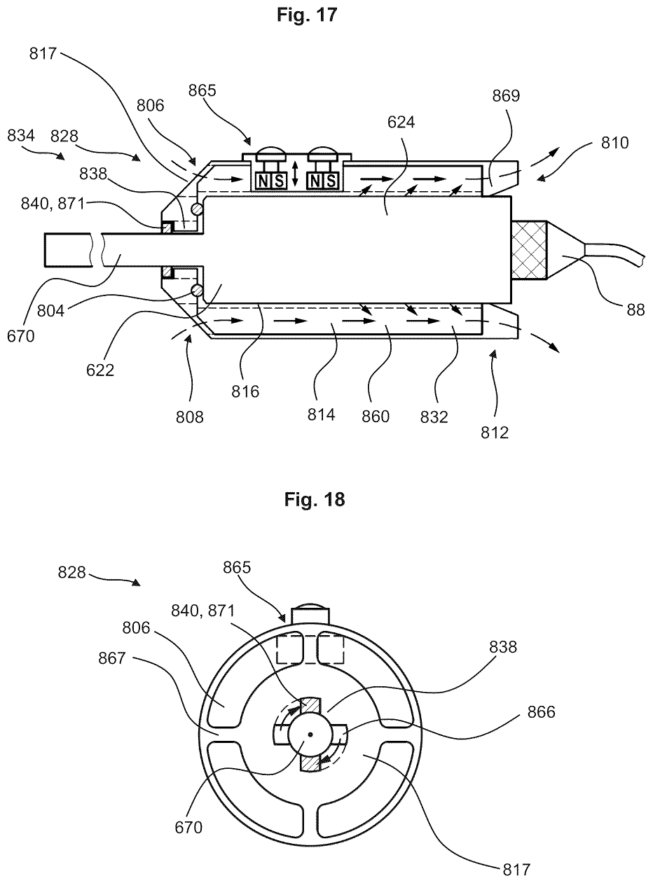

FIG. 17 shows a schematic representation of a further handling module;

FIG. 18 shows a schematic representation of the handling module viewed from a distal side;

FIG. 19 shows a schematic representation of the handling module viewed from a proximal side;

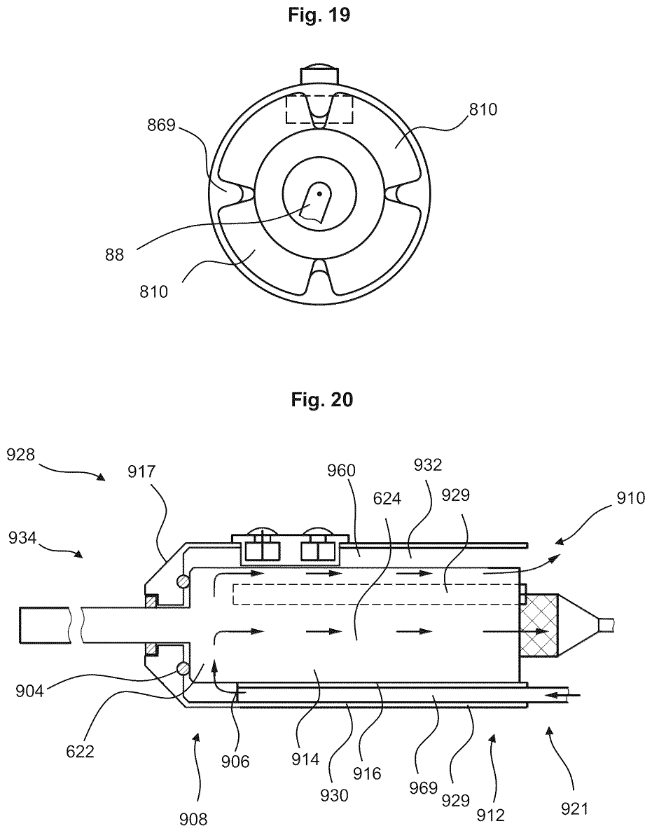

FIG. 20 shows a schematic representation of a further handling module;

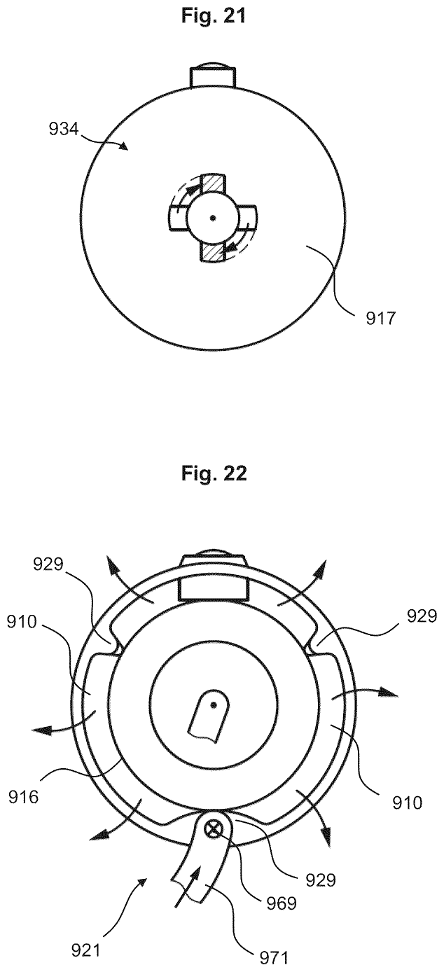

FIG. 21 shows a schematic representation of the handling module viewed from a distal side;

FIG. 22 shows a schematic representation of the handling module viewed from a proximal side;

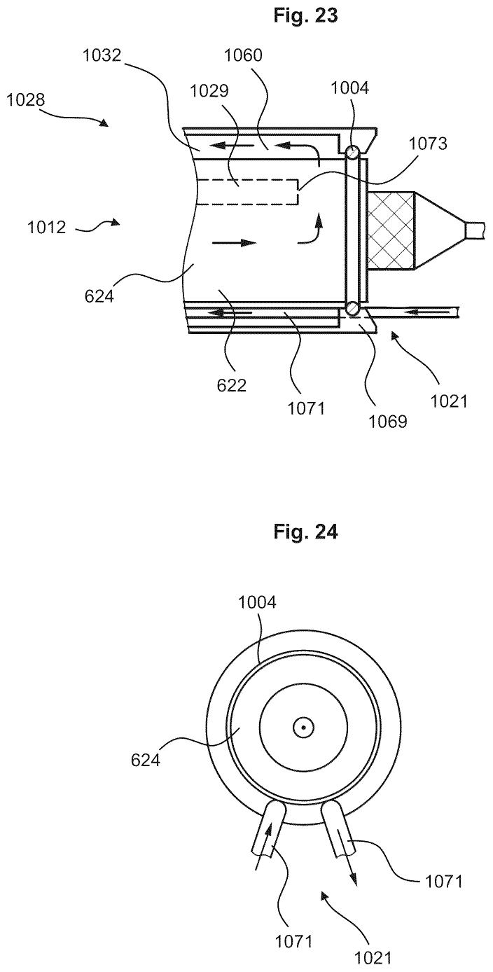

FIG. 23 shows a schematic representation of a proximal section of a further handling module;

FIG. 24 shows a schematic representation of the handling module viewed from the proximal side;

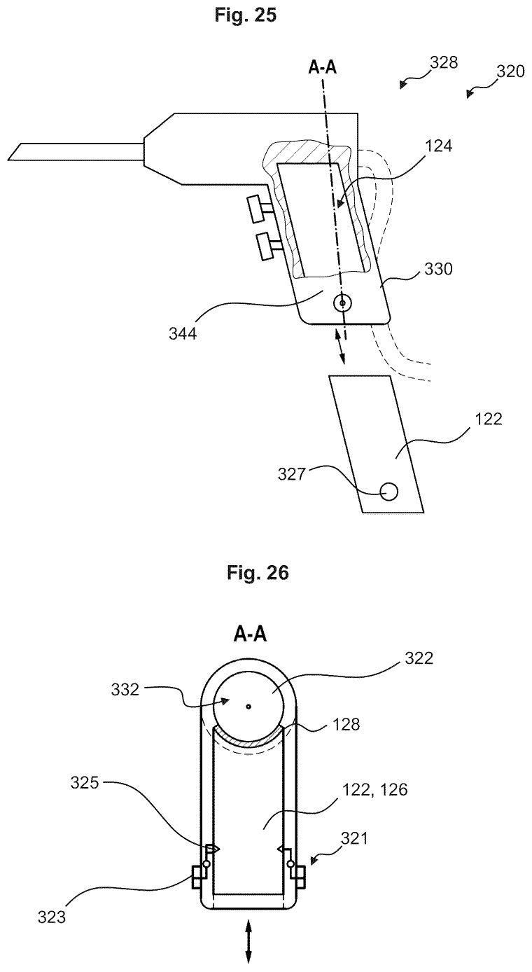

FIG. 25 shows a schematic representation of the endoscope;

FIG. 26 shows a sectional view of the proximal side of the handling module;

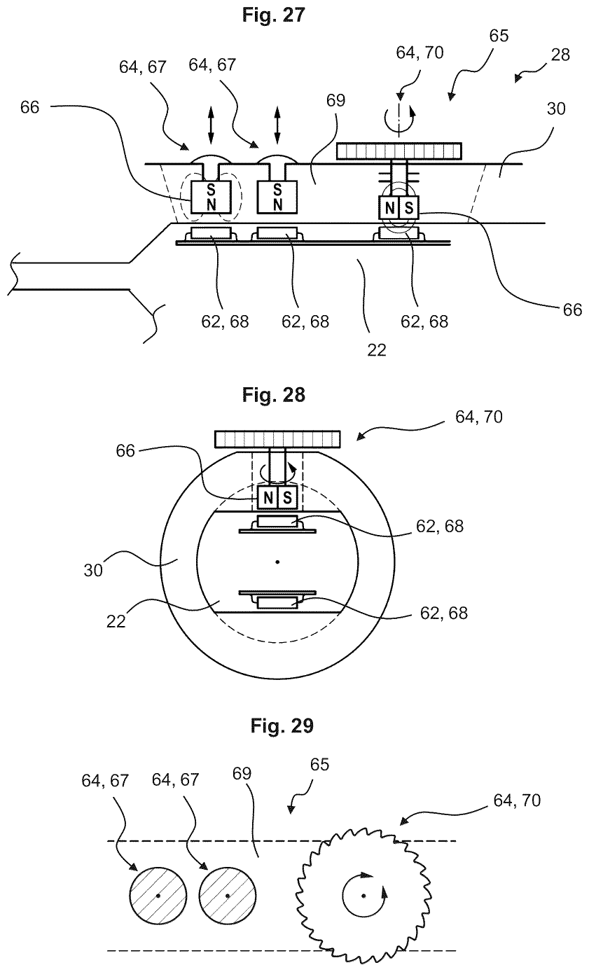

FIG. 27 shows a schematic detailed representation of the operating assembly in a side view;

FIG. 28 shows a schematic view of the operating assembly from the proximal side;

FIG. 29 shows a schematic plan view of the operating assembly;

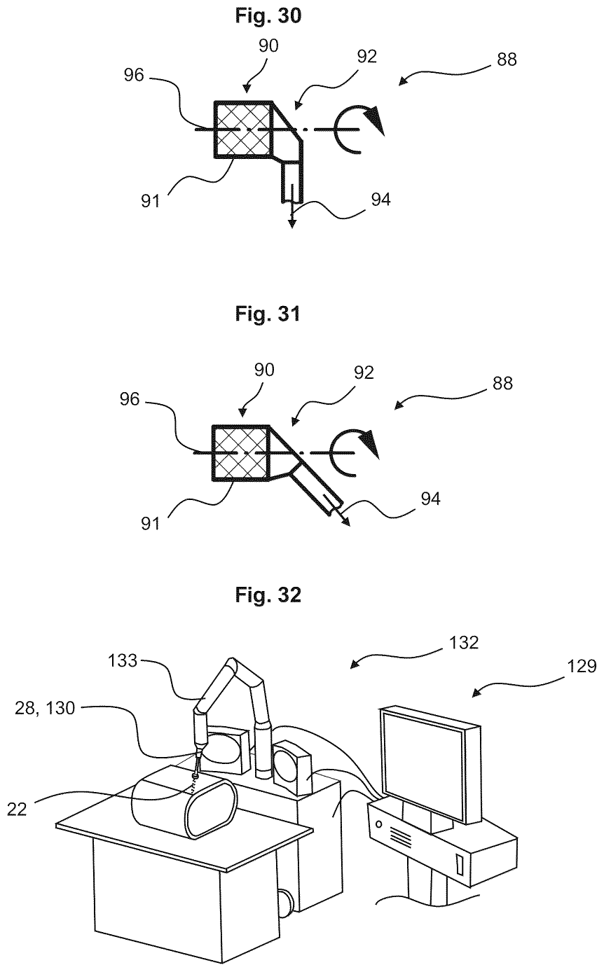

FIG. 30 shows a schematic representation of a supply cable;

FIG. 31 shows a schematic representation of a further supply cable;

FIG. 32 shows a schematic representation of a medical robot; and

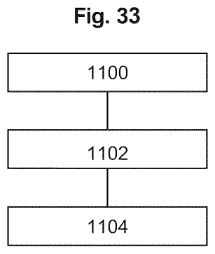

FIG. 33 shows a schematic flow diagram of a method.

FIG. 1 shows a system 10 comprising a supply unit 12, a display unit 14, an endoscope 20, and a sterile single-use product 134. The endoscope 20 comprises a base module 22. The base module 22 has a housing 24 and control electronics 26 which are enclosed in a gas-tight manner.

Furthermore, the endoscope 20 comprises a handling module. The handling module 28 is configured to make the base module 22 handleable. In addition, the handling module 28 comprises a main body 30 which defines an accommodation space 32 for the base module 22. To establish a handleable state, the base module 22 is introducible into the accommodation space 32 of the handling module 28 by a user. In the illustrated case, the base module 22 is introduced into the accommodation space, and the endoscope 20, in particular the base module 22, is handleable.

The user can generally remove the base module 22 from the handling module 28 and introduce a further base module into the accommodation space 32 of the handling module 28. This could make the additional base module handleable.

The handling module 28 and the base module 22 together form a securing mechanism 34, shown only schematically, by means of which the base module 22, in particular the housing 24, is releasably securable to the handling module 28. In particular, the base module 22 is releasable from and/or securable to the handling module 28 without tools.

The handling module 28 is configured to make the base module 22 handleable and operable by the user. An operable state can be established by introducing the base module 22 into the accommodation space 32. In this regard, the main body 30 comprises a handle 44. The handle 44 has an ergonomic shape not shown in FIG. 1. In the handleable state, the handle 44 completely encompasses the housing 24. A longitudinal axis of the handle 44 corresponds to a longitudinal axis of the accommodation space 32. These longitudinal axes coincide accordingly on the axis 86 shown in FIG. 1. In the operable state, a longitudinal axis of the base module 22 also coincides with the longitudinal axis of the accommodation space 32. A main extension direction of the base module 22 and/or of the accommodation space 32 and/or of the handle 44 runs parallel to the aforementioned longitudinal axes or to the axis 86.

The handling module 28 is made at least partially and in particular at least largely from plastics material. In addition, the main body 30 is manufactured by injection molding. Alternatively, the main body 30 can be manufactured additively or by an additive manufacturing process. In particular, the handling module 28 is manufactured inexpensively and/or with low material expenditure. The handling module 28 is intended for single use in the present case. It could be described as single-use. In addition, the handling module 28 and/or the base module 22 are configured to be autoclaved and/or sterilized separately. In particular, the handling module 28 is configured to be autoclaved and/or sterilized separately from the base module 22.

Furthermore, the handling module 28 comprises operating elements 64, two of which are shown as examples in FIG. 1. It is understood, however, that a different number of operating elements 64 may be provided. The operating elements 64 are arranged on the handle 44 and/or are part thereof. The user can operate the base module 22 using the operating elements 64. In particular, the operating elements 64 have an indirect effect in this case. In other words, the operating elements 64 are not part of the base module 22, but act on it in a way that enables the user to operate the base module 22 by operating the operating elements 64.

The base module 22 comprises in the present case a shaft 70 having a distal section 72 and having a proximal section 74, wherein the proximal section 74 is attached to the housing 24. The distal section 72 comprises a camera 78 and/or a light source 80. The shaft 70 is, for example, 40 cm long and has, by way of example, a diameter of 2 cm. A longitudinal axis of the shaft 70 coincides with the longitudinal axis of the base module 22, the longitudinal axis of the accommodation space 32, the longitudinal axis of the handle 44 and the axis 86.

The shaft 70 comprises a heat pipe (not shown) and lines (not shown). The heat pipe and/or the lines extend along the shaft 70, in particular from the distal section 72 to the proximal section 74. The heat pipe and/or the lines protrude beyond the proximal section 74 into the housing 24 of the base module 22 and/or into the handling module 28 or are connected and/or connectable to the housing 24 and/or the base module 22 via suitable connections. The lines are connected to the control electronics 26. The lines are configured to connect controllable and/or electronic elements to the control electronics 26. In particular, the lines connect the camera 78 and/or the light source 80 to the control electronics 26. The heat pipe conducts waste heat generated during the operation of the camera 78, the light source 80 and/or the other electronic components along the shaft 70 to the housing 24 of the base module 22.

The distal section 72 comprises an optical element 82 that defines a main viewing direction 84. The main viewing direction 84 is inclined with respect to the axis 86 and is in particular 45°. The optical element may, for example, be a lens that is installed at a certain angle relative to a longitudinal axis of the shaft 70.

The camera 78 comprises an image sensor, not shown, which may be, for example, a CCD chip or a CMOS chip. In some embodiments, the camera 78 may be a stereo camera and/or comprise two image sensors. The camera 78 is configured to generate image data.

The light source 80 comprises a luminous element not shown. The light source 80 may also comprise multiple luminous elements. The luminous element is, for example, an LED, in particular a white light LED. The light source 80 may be multimodal and provide illumination light optionally in different spectral ranges. In addition, the light source 80 may be configured to provide illumination light continuously and/or in a pulsed manner. The light intensity of the light source 80 is adjustable automatically and/or by the user. The control electronics 26 are configured to supply the light source 80 with application-specific and/or operating state-specific electrical energy. The lines are configured to transmit the image data from the camera 78 to the control electronics 26.

In other embodiments, the supply unit 12 may also provide illumination light for the endoscope 20. For example, an illumination device may be provided that provides illumination light for imaging. The endoscope 20 can be couplable and/or coupled to the illumination device via a fiber optic cable. The endoscope 20 may in this case have a suitable light guide element in its shaft 70, by means of which illumination light can be guided to the distal end 72. The light source 80 may in this case be omitted or provided additionally. In other embodiments, a separate illumination device and/or illumination light source may be provided.

In the present case, the base module 22 is also connected to a supply cable 88. In some embodiments, the supply cable 88 may be permanently attached to and/or be part of the base module 22. In the present case, the supply cable 88 is releasable. The supply cable 88 is a coaxial cable and is configured to be sterilizable and/or autoclavable. The supply cable 88 is rotatable relative to the base module 22 in a connected state. In particular, the supply cable 88 is rotatable about the longitudinal axis of the base module 96. The base module 22 comprises a supply cable storage system configured for this purpose (not shown). The supply cable 88 is connected to the supply unit 12 and is configured to supply the base module 22, in particular the control electronics 26, with electrical energy and to enable data exchange. In particular, image data generated by the camera 80 is transmitted to the supply unit 12 via the supply cable 88. The supply unit 12 is also connected to the display unit 14. The display unit 14 is configured to display the image data to the user.

The user can use the endoscope 20 for a diagnostic and/or therapeutic action. For example, he can feed the shaft 70 into a body cavity of a patient in order to be able to view the anatomical structure 16. The anatomical structure 16 is displayed to the user by the display unit 14. It may be the case that the user is restricted by the main viewing direction 84 of the endoscope 20 when performing the diagnostic and/or therapeutic action. Consequently, the user can provide a different base module that defines a different main viewing direction. To do so, the user can remove the base module 22 from the handling module 28 and introduce and releasably secure the other base module into the accommodation space of the handling module 28.

The sterile single-use product 134 comprises an outer packaging 136 and a further handling module 29. In the exemplary case, the further handling module 29 is different from the handling module 28. The two handling modules 28, 29 implement different functionalities in the handleable state of the base module 22. The further handling module 29 is packaged in a sterile state in the outer packaging 136.

It is understood that the handling module 28 may also have been packaged in an analogous sterile outer packaging prior to its use in the endoscope 20. The handling module 28 is advantageously part of such a single-use product. It is only unpacked by the user or other clinical staff immediately before use, for example in preparation for an operation, and is therefore sterile. There is no need for additional sterilization or draping. The user can then introduce the base module 22 into the handling module 28, thereby making the base module 22 handleable.

Analogously, the user can remove the additional handling module 29 from the outer packaging 136 and/or provide said additional handling module. The handle of the handling module 28′ is, for example, a pistol grip, which may be advantageous in terms of the action. In addition, the user can release the base module 22 from the handling module 28 and remove the base module 22 from the handling module 28. The user can then introduce the base module 22 into the further handling module 29, in particular into an accommodation space thereof. The handling module 28 can be disposed of or thrown away by the user. In addition, the base module 22, in particular together with the supply cable 88, can be sterilized and/or autoclaved before the user introduces the base module 22 into the handling module 28′. Furthermore, the user can releasably secure the base module 22, in particular the housing of the base module 22, to the further handling module 28 before an endoscope thus produced is used.

In the present case, the shaft 70 is firmly connected to the housing 24. The shaft 70 can be formed integrally with the housing 24. Alternatively or additionally, the shaft 70 can be welded or glued to the housing 24, for example. A connection between the shaft 70 and the housing 24 may be vapor-tight.

In some embodiments, the shaft 70 may be designed as an interchangeable shaft. The shaft 70, designed as an interchangeable shaft, can in this case be connected to the housing 24 without tools. In this way, different interchangeable shafts can be used, which, for example, have different cameras, different light sources, different lengths, different diameters, different materials, etc. and/or define different main viewing directions. In general, different interchangeable shafts may be provided for different applications.

FIG. 2 shows a schematic view of the distal end 72 of the shaft 70 in a side view. Some details can be seen in more detail here. The camera 78 comprises the optical element 82 and an image sensor 148. The optical element 82 defines the main viewing direction 84 of 45°, which is inclined relative to the longitudinal axis of the shaft 70 or the axis 86. In addition, the shaft 70 comprises the light source 80. In addition, the shaft 70 comprises a heat pipe 152. The heat pipe 152 extends at least in a close range of the camera 78. In addition, the shaft 70 comprises two electronic elements 150, 151. These may be, for example, circuit boards, control processors, circuits, sensors or other components.

FIG. 3 shows a schematic representation of the base module 22. As mentioned, the system 10 comprises the supply cable 88, which is connectable to the base module 22. The base module 22 and the supply cable 88 together form a plug connector 90. The supply cable 88 is a coaxial cable 92 and, when connected to the base module 22, is rotatable relative to the base module 22. A part, not shown, of the supply cable 88 may be connected to the supply unit 12. A plug connector may also be provided for connecting the supply cable 88 to the supply unit 12. This plug connector can also establish a rotatable connection. Alternatively, as schematically indicated in FIG. 1, the connection between the supply cable 88 and the supply unit 12 may be designed differently than the connection between the base module 22 and the supply cable 88, for example as a plug with multiple contacts arranged next to one another and/or as a plug with a preferred plug-in orientation.

The base module comprises multiple actuating elements 62, which are all provided with the same reference numeral for reasons of simplicity. The actuating elements 62 may be designed differently and/or control different functionalities of the base element 22. For example, they can be used to actuate the camera 78 and/or to actuate the light source 80 and/or to adjust a light intensity and/or light color.

FIG. 4 shows a schematic representation of the handling module 28 and the base module 22 before the base module 22 is introduced into the accommodation space 32 of the handling module 28. The handling module 28 has multiple operating elements 64 which are arranged on an outer side of the main body 30. For the sake of simplicity, the operating elements 64 are all provided with the same reference numeral, but may be designed differently. For example, at least one of the operating elements 64 may be designed as a push button. Furthermore, at least one of the operating elements 64 may be designed as a rotary knob. In addition, at least one of the operating elements 64 may be designed as a slider. The operating elements 64 may be part of an operating assembly 65. The operating assembly 65 may comprise its own housing which is connected to the main body 30. The operating assembly 65 may be manufactured separately from the main body. In the present case, for example, the main body 30 is an injection-molded part into which the operating assembly 65 is installed.

In other embodiments, the operating elements 64 may be formed integrally with the main body 30. These can be connected to adjacent material, for example, via webs and/or film hinges and/or the like and can be manufactured in a single injection molding step.

The operating elements 64 are arranged such that they act on the actuating elements 62 in at least one introduction position of the base module 22. In particular, a unique, in particular unambiguous, assignment may be provided so that exactly one specific functionality of the base module 22 can be controlled by means of one operating element 64 in each case, by the operating element 64 acting on an actuating element 62 associated with the functionality.

As can be seen in FIG. 3, actuating elements 62 are arranged at different circumferential positions of the housing 24 of the base module 22. In the case shown, two groups of actuating elements 62 which are identical and/or analogous to one another are arranged at opposite positions of the housing 24. This makes it possible to introduce the base module 22 into the accommodation space 32 in two different orientations, wherein for each of the two orientations one of the groups of actuating elements 62 interacts with the operating elements 64. If, as in the present case, an angular main viewing direction 84 is provided, two different viewing directions can be implemented with the same base module 22 for a predefined manual holding position of the handling module 28, in the present case, for example +45° and −45°, depending on the orientation.

The accommodation space 32 extends, for example, along 90% of a length of the handling module 28. FIG. 5 shows a simplified schematic view of the handling module 28 viewed from its proximal side 98. Reference is also made below to FIG. 5. The main body 30 forms on its proximal side 98 an opening 100 through which in particular the base module 22 is introducible into the accommodation space 32. In addition, the handling module 28, in particular the main body 30, forms a distal opening 154 on its distal side 156. The distal opening 154 is designed such that the shaft 70 can be drawn out through the distal opening 154.

The accommodation space 32 has a circular cross-section. The housing 24 of the base module 22 has a circular cross-section. In an introduced state, the housing 24 is arranged concentrically with the accommodation space 32. The accommodation space 32 has a cylindrical section. The housing 24 has a cylindrical section. The cylindrical section of the housing 24 is arranged in the introduced state in the cylindrical section of the accommodation space 32.

In principle, base modules may also be integrated into an accommodation space for handling modules during factory assembly. In this context, the user may be a user other than the end user. For example, the user may be an assembler in a factory. It is also conceivable for the user to operate a machine that is configured to introduce base modules into an accommodation space for handling modules.

FIG. 6 shows a schematic representation of an endoscope 220. The endoscope 220 is generally designed analogously to the endoscope 20 described above. Below, the main focus is on differences. The endoscope 220 comprises a base module 222 and a handling module 228. The base module 220 is introducible into an accommodation space 232 of the handling module 28 and is shown in an introduced state in FIG. 6. The base module 222 is introducible into the accommodation space 232 from a proximal side of the handling module 228 through an opening.

The handling module 228 has a handle 244. In the present case, the handling module 228 has a main body 230 which forms the handle 244. The handle 244 is arranged, at least in sections, distally with respect to a housing 224 of the base module 222. The 244 has a first diameter 252. The housing 224 has a second diameter 254. The first diameter 252 is smaller than the second diameter 254.

FIG. 6 shows the endoscope 220 in a user's hand. It can be seen that the section of the handle 244 or the handle 244 arranged distally with respect to the housing 224 is, as a whole, pen-shaped. “Pen-shaped” means that the handle 244 is grippable by the user as if it were a pen being gripped for writing. This allows the endoscope 220 to be guided very precisely. Small movements can therefore be carried out in a controlled manner. The endoscope 220 is suitable for applications in neurosurgery, for example.

FIG. 7 shows a schematic representation of an endoscope 320. The endoscope 320 is generally designed analogously to the endoscopes 20, 220 described above. Below, the main focus is on differences. The endoscope 320 comprises a base module 322 and a handling module 328. The base module 320 is introducible into an accommodation space 332 of the handling module 328 and is shown in an introduced state in FIG. 7.

The handling module 328 comprises a handle 344. The handle 344 is arranged, at least in sections, proximally with respect to the housing 324. In the present case, the handle 344 encloses the housing 324 on a proximal side. According to other embodiments not shown, the main body according to FIG. 5 may form on its proximal side an opening through which in particular the base module 22 is introducible into the accommodation space. A longitudinal axis 346 of the handle 344 is inclined with respect to a longitudinal axis 348 of the accommodation space 342. The longitudinal axis 348 of the accommodation space 342 and the longitudinal axis 346 of the handle 344 enclose, for example, an angle of 100°. The 344 handle is a pistol grip. In addition, the handling module 328 comprises two operating elements 364, one of which is shown in more detail in FIG. 8. The operating elements 364 are arranged on the handle 344. The operating elements 364 can be operable, for example, with an index finger and/or a middle finger and/or a ring finger when the handle 344 is gripped, in particular in the manner of a trigger.

FIG. 8 shows a further schematic representation of the endoscope 320 from FIG. 7 with a detailed view of the operating element 364 of the endoscope 320. The operating element 364 is coupled to an actuating mechanism 358. The actuating mechanism 358 may also be part of the actuating element 364. The operating element 364 may have at least two operating states. One of the operating states is shown in solid lines, while another operating state is shown in dashed lines. By operating the operating element 364, the actuating mechanism 358 is actuated and, in particular, moved into another actuating state. The actuating mechanism 358 comprises, in particular, a lever mechanism 360 which is configured to guide a movement of the operating element 364 to a close range of an actuating element 362 of the base module 322. The movement may be caused by the user, for example by exerting a pressure force on the operating element 364. The operating element 364 can be spring-loaded, in particular into a non-actuated position.

The actuating mechanism 358 can be coupled to a trigger element 370. The trigger element 370 may also be part of the operating element 364. The trigger element 370 comprises a magnet 366 in the present case. The magnet 366 may be a permanent magnet, for example. By operating, in particular pressing, the operating element 364, the trigger element 370 can be moved away from the actuating element 362. As a result, the operating element 364 can act on the actuating element 362 in such a way that an actuation of the actuating element 362 can be effected, in particular by the user.

In the present case, the actuating element 362 comprises a Hall sensor 368. The Hall sensor 368 is configured to detect a movement or a position and/or a change in position of the trigger element 370. For this purpose, the Hall sensor 368 senses a magnetic field of the magnet 366 in the present case.

FIG. 9 shows a schematic representation of the handling module 328 in a view parallel to a longitudinal axis of the accommodation space 332. A main body 330 of the handling module 328 has two main body parts 340, 342 which are connected to one another in an articulated manner. The main body parts 340, 342 are movable relative to each other to open and close the accommodation space 332. In an open state of the accommodation space 332, the base module 322 is introducible into the accommodation space 332. In a closed state of the accommodation space 332, the housing 324 of the base module 322 is secured to the handling module 328.

The main body 330 has a hinge 350. The hinge 350 may, for example, be designed as a hinge strip that extends parallel to a longitudinal axis of the accommodation space 332. In some embodiments, the hinge 350 may be a film hinge. The hinge 350 can in this case be manufactured in a single manufacturing step together with the two main body parts 340, 342, in particular by means of injection molding and/or by means of additive manufacturing. Preferably, the main body parts 340, 342 are made of plastics material.

The handling module 328 has a securing mechanism 334. The securing mechanism 334 in the present case comprises a locking element 336 which can engage in a locking receptacle 338 in order to establish a locking state. The locking element 336 and the locking receptacle 338 are each provided on one of the main body parts 340, 342. The securing mechanism 334 is a clip closure. After placing the base module 322 into the accommodation space 332, the user can fold the two main body parts 340, 342 and close the securing mechanism 334. The base module 322 is then secured in the handling module 328.

In some embodiments, the main body 330 may be provided on an inner side with at least one elastic heat-conducting element 352, which can be formed, for example, from a heat-conducting elastomer. In the present case, such an elastic heat-conducting element 352 is provided on each of the main body parts 340, 342. In the introduced state of the base module 322, the at least one heat-conducting element 352 contacts the housing of the base module 322. As a result, waste heat can be dissipated via the at least one heat-conducting element 352. In addition, the at least one heat-conducting element 352 can clamp the base module 322, whereby the latter is securely held in the accommodation space 332.

FIG. 10 shows the handling module 328 in a state in which the accommodation space 332 is closed. The two main body parts 340, 342 are closed and locked together by means of the securing mechanism 334.

FIG. 11 shows a schematic representation of a securing mechanism 34. For example, this could be the securing mechanism used in FIG. 1. The securing mechanism 34 for the endoscope 20 and its components are described below. However, a securing mechanism of this type may also be used in other embodiments described herein.

The base module 22, in particular its housing 24, is introduced into the accommodation space 32 of the handling module 28 in such a way that a free space 160 is present between the housing 24 and the main body 22. The free space 160 is hollow cylindrical in shape. Due to the free space 160, the housing 24 of the base module 22 does not rest with its entire outer surface against an inner wall of the main body 30. FIG. 12 shows a schematic representation of the securing mechanism 24 viewed from a proximal side. In the following, reference is also made in part to FIG. 12.

The securing mechanism 34 comprises a pretensioned and/or pretensionable pretensioning element 36. In the present embodiment, the pretensioning element 36 is attached to a cable base 166. The cable base 166 may be part of the supply cable 88. The pretensioning element 36 can be removable from the base module 22 together with the supply cable 88. In other embodiments, the pretensioning element 36 may be permanently attached to the base module 22. This is possible both in variants in which the supply cable 88 is permanently attached to the base module 22 and in variants in which an interface between the base module 22 and the supply cable 88 is releasable. For example, in these cases, the cable base 166 may be replaced by a base element of the base module 22 and/or a projection of the housing 24 which carries the pretensioning element 36.

The pretensioning element 36 is designed as a spring element. The pretensioning element 36 may have multiple spring-loaded arms and/or arm sections. The pretensioning element 36 may be radially deformable. In particular, an outer diameter of the pretensioning element 36 can be reduced by the application of force. The pretensioning element 36 may be designed in the manner of a disc spring, in particular as a multi-slotted disc spring. Alternatively, the spring element 36 may have individual arms. For example, two of these arms can be shown in FIG. 9. Preferably, at least two, at least three, at least four, at least five or at least six arms are present.

The securing mechanism 34, in particular the handling module 28, comprises a holding element 38 which is configured to releasably secure the housing 24 to the handling module 28 by engaging behind it. In particular, the pretensioning element 36 is configured to releasably secure the housing 24 to the handling module 28 by engaging behind the holding element 38.

The pretensioning element 36 is shown in FIG. 11 in two different positions. The position shown further to the right (solid lines) is a position in a securing state. The pretensioning element 36 is in a secured position. The position shown just to the left (dashed lines) corresponds to a state of the pretensioning element 36 during introduction and/or removal from the accommodation space 32. The pretensioning element 36 is in a release position.

To introduce the base module 22, the user compresses the pretensioning element 36 radially to move it into the release position. When introducing the base module 22 into the accommodation space 32, the pretensioning element 36 can then pass the holding element 38. The pretensioning element 36 then relaxes into its secured position, engaging behind the holding element 38. The pretensioning element 36 exerts a force on a distal contact surface of the holding element 38, whereby the base element 22 is pressed into the accommodation space 32 and/or held in the accommodation space 32. If a force acting in the proximal direction is exerted on the base module 22 in the introduced and secured state, the pretensioning element 36 counteracts this force and holds the base module 22 in the handling module 28.

In addition, the handling module 28 and the base module 22 together form an alignment aid 56 which defines a preferred position and/or a preferred orientation of the housing 24 relative to the handling module 28. In particular, the alignment aid 56 comprises two oblique sections 162, 163 which extend along the circumference of the handling module 28 and of the base module 22, in particular of the housing 24, and are configured to interact with one another, in particular by mechanical contact, such that the base module 22 assumes an axial preferred position in a handleable state. Furthermore, the alignment aid 56 is configured to center the base module 22 such that the longitudinal axis of the base module 22 coincides with the longitudinal axis of the handle 44. The oblique sections 162, 163 are conical in the present case. They comprise conical surfaces which rest against one another and which together center the base module 22.

In addition, the handling module 28 has a sealing element 104 which seals against the base module 22 in the operable state. The sealing element 104 is arranged proximally with respect to the alignment aid 56. The sealing element 104 is, for example, an O-ring. The sealing element 104 is preferably made of a, in particular elastic, deformable material, for example of rubber and/or an elastomer. In the present case, the pretensioning element 36, when introduced, presses the base module 22 or its housing 24 against the sealing element 104.

In the present case, the main body 30 further comprises a support element 169. The support element 169 supports the housing 24 inside the accommodation space 32. In the illustrated case, the support element 169 rests against a proximal end of the housing 24 of the base module 22 on an outer circumferential surface of the housing 24. The support element 169 defines an inner diameter that at least substantially corresponds to an outer diameter of the housing 24. The support element 169 may be annular. Alternatively, multiple support elements may be provided at different circumferential positions.

In embodiments in which the shaft 70 is designed as an interchangeable shaft, an interface 71 may be provided between the shaft 70 and the housing 24. Such an interface 71 is shown in FIG. 9 as an optional component in dashed lines. Such an interface 71 may be arranged such that it is located within the accommodation space 32 when the base module 22 is introduced. For example, the interface 71 is located proximally in relation to the distal opening 154 of the main body 30. As a result, the interface 71 is protected by the handling module 28.

FIG. 13 shows a further embodiment of a handling module 428 and a base module 422. The design is similar to that shown in FIG. 7-9. In contrast, however, a sealing element 404 is arranged distally from the accommodation space 432. The sealing element 404 can seal against a shaft 470. In the present case, the sealing element 404 is an O-ring and/or preferably made of a, in particular elastic, deformable material, for example of rubber and/or an elastomer.

If the shaft 470 is designed as an interchangeable shaft and can be connected to a housing 424 of the base module 422 via an interface 471, the interface may be arranged proximally with respect to the sealing element 404. The interface 471 can thus be protected by the handling module 428. In particular, the interface 471 is tightly enclosed from at least one distal side.

FIG. 14 shows a schematic representation of another embodiment of a securing mechanism 624 viewed from a proximal side. The embodiment is similar to the embodiment shown in FIG. 12. Therefore, the differences shall primarily be discussed below. The securing mechanism comprises a rotatable securing element 640 which is rotatably arranged on a proximal end surface of a base module 622. In particular, the rotatable securing element 640 is rotatably mounted about a longitudinal axis 696 of the base module 622. The longitudinal axis 696 coincides with a longitudinal axis of a handling module 628 in an introduced state of the base module 622. In the present embodiment, the securing element 640 is attached to the cable base 166 similarly to FIG. 12. The cable base 166 may be part of the supply cable 88. Further features and alternative embodiments may be similar to those of the supply cable 88 of FIG. 12. In particular, the securing element 640 can be provided instead of the pretensioning element 36. In particular, in such a case, the base module 622 can be the same as the base module 22. The securing element 640 can in particular be provided on the cable base 166 and the securing element 640 can be removable together with the cable base 166. If, for example, the base module 22, 622 is to comprise the pretensioning element 36 instead of the securing element 640 in order to make it handleable with the handling module 28, the cable base 166 can be connected to the base module 22, 622.

In addition, the securing mechanism comprises two holding elements 638, which are formed by the handling module 628. The holding elements 638 are arranged on opposite sides of the handling module 628. The holding element 638 is configured to releasably secure a housing 624 of the base module 622 to the handling module 628 by engaging behind it. In particular, the securing element 640 is movable by rotation between a securing state and a releasing state. In FIG. 14, the securing element 640 is shown in solid lines in the release state. In this state, the base module 622 is introducible into an accommodation space 632 and/or drawn out from the accommodation space 632. The base module 622 is introducible into the accommodation space 632 in such a way that a lateral section of the securing element 640 engages behind one of the holding elements 638 in the securing state. The securing state is shown by dashed lines. In particular, the securing state and/or the release state can be achieved without tools.

The user can introduce the base module 622 into the accommodation space and rotate the securing element 640 such that the securing element 640 engages behind at least one of the holding elements 638 in order to establish the securing state. Furthermore, the user can rotate the securing element 640 such that the release state is established and remove the base module 622 from the handling module 628. In addition, the user can release the supply cable 88 together with the cable base 166 and the securing element 640 from the base module 622. Subsequently, the user can connect the supply cable 88 and the cable base 166 of FIG. 12 comprising the pretensioning element 36 to the base module 622. This allows the user to make the base module 622 compatible with the handling module 28.

A further aspect of the securing mechanism 624 is illustrated in part with reference to FIG. 15. The securing element 640 comprises a pretensionable pretensioning element 636. In particular, the securing element 640 can be the pretensioning element 636. The pretensioning element 636 is elastically deformable in the axial direction. FIG. 15 shows a schematic side view of the holding element 638. The holding element 638 may define, in sections, a guideway 643 which is angled with respect to a plane perpendicular to the longitudinal axis 696. The guideway 643 is configured to deform the pretensioning element 636 in the axial direction upon rotation of the securing element 640. In addition, the holding element 638 comprises a locking section 645 which is designed as a locking recess, in particular as a groove. In the securing state, the locking section 640, or the locking recess, at least partially encloses the pretensioning element 636. In this state, the pretensioning element 636 is in an axial preferred position. To be moved out of the preferred position, an axial force must be applied to the pretensioning element 636, preferably by the user. The pretensioning element 636 deforms in the axial direction during rotation by being guided on the guideway. This allows a tension and/or potential energy of the pretensioning element 636 to be generated. If the securing element 640 and/or the pretensioning element 636 is rotated beyond the angled guideway 643, the pretensioning element 636 moves or snaps into the preferred position, in which the tension or potential energy is lower than on the angled guideway 643. For releasing the securing mechanism 634, the pretensioning element 636 can be deformed in the axial direction, in particular by a user, such that the securing element 640 can be rotated beyond the locking section 645. In particular, to release the securing mechanism 634, the user can axially deform the pretensioning element 636, or press on the pretensioning element 636 from the proximal side, and move the securing element 640 into the release state.

It is understood that the shown embodiments of the securing mechanisms may be combined as desired with base modules and/or handling modules. In particular, the securing mechanisms relate to features that are to be understood independently of other features of the base modules and/or handling modules.

FIG. 16 shows a view of a transverse section through a further embodiment of a base module 722 and a handling module 728. The base module 722 and the handling module 728 together form an alignment aid 756. The alignment aid 756 comprises four guide lamellae 758 which are formed on a radial inner side of the handling module 728. The guide lamellae 758 protrude into an accommodation space 732 of the handling module 728. In addition, the alignment aid 756 comprises four guide grooves 761. The guide grooves 756 are each designed to correspond to one of the guide lamellae 758. In particular, the guide grooves are designed as recesses in a housing 724 of the base module. The alignment aid 756 allows a radial preferred position of the base module 722 to be defined by the guide lamellae 758 contacting all guide grooves 761. In particular, the base module 722 can be centered, or the base module 722 is arranged centered in the accommodation space 732 in the introduced state. The guide lamellae 758 also protrude into the accommodation space 732 in such a way that, in the introduced state of the base module 722, a free space 760 remains between the housing 724 and the handling module 728. In addition, the guide grooves 761 at least partially accommodate the guide lamellae 758. In particular, the guide lamellae 758 can contact side walls, in particular all side walls, of the guide grooves 761. This allows a preferred rotational position to be defined. Furthermore, the guide lamellae 758 and the guide grooves 761 can improve the guiding properties of the base module 722 when the base module 722 is being introduced into the accommodation space 732.

FIG. 17 shows a further embodiment of a handling module 828 in a schematic representation, wherein the base module 622 is shown in an introduced state, or ready-to-use state, in an accommodation space 832 of the handling module 828. The handling module 828 is very similar at least to the handling module 28. Therefore, the differences shall primarily be discussed. The present embodiment of the handling module 828 is described below with the aid of FIG. 18 and FIG. 19. FIG. 18 shows a schematic representation of the handling module 828 as viewed from a distal side of the handling module 828. FIG. 19 shows a schematic representation of the handling module 828 as viewed from a proximal side of the handling module 828.

The handling module 828 comprises four air inlets 806 on a distal section 808 of the handling module 828. The air inlets 806 are designed as openings to the accommodation space 832 of the handling module 828. The openings are apertures through a distal end surface 817 of the handling module 828. The air inlets 806 may collectively extend over a majority of a circumference of the distal end surface 817 of the handling module 828. In the illustrated case, the air inlets 806 extend over 90% of the circumference of the distal end surface 817 of the handling module 828. Alternatively, different designs of the air inlets 806 may be provided. For example, any number of air inlets, in particular two, three, five, six or seven air inlets, may be provided. The air inlets may collectively extend, for example, over 30% to 99%, in particular over 50% to 95%, preferably over 70% to 90%, of the circumference of the distal end surface 817 of the handling module. In particular, the extension of the air inlets may be selected with respect to the power of the base module. If a greater cooling effect is desired because the base module has a higher power during operation, a larger extension of the air inlets can be provided. In other embodiments not shown, the air inlets may be arranged at least partially on a radial outer surface of a handling module. In addition, the air inlets may be arranged at least partially on a radial outer surface of a handling module and/or at least partially on a distal end surface 817 of the handling module. Referring again to the embodiment shown, the remaining part of the distal end surface 817 comprises four webs 867 which extend in particular radially between the air inlets 806.

Furthermore, the handling module 828 comprises four air outlets 810 on a proximal section 812 of the handling module 828. The air outlets 810 are interrupted by four support elements 869. The support elements 869 together form a ring, viewed from the proximal side, which tapers in the axial direction. In particular, the support elements 869 together form a cone. The section of the cone with the smallest diameter defines an inner diameter that at least substantially corresponds to an outer diameter of the housing 624. In the introduced state, the support elements 869 are configured to guide the base module 622 axially and to define a radial preferred position. In particular, the support elements 869 are configured to center the base module 622. In the handleable state, there is a free space 860 between the base module 622 and the handling module 828. In other embodiments of a handling module, the proximal side of the base module 722 may be enclosed. In this case, air outlets can be formed, corresponding to the air inlets, as apertures at least partially through a proximal wall and/or at least partially through a radial outer surface of the handling module.

The air inlets 806 and the air outlets 810 are connected to each other, in particular via the free space 860. The connection between the air inlets 806 and the air outlets 810 may also be defined as an air duct 814. The air duct is hollow cylindrical in shape. In particular, in a central section of the housing there is primarily no contact with the handling module 828. A radial inner surface 816 of the air duct is defined by the housing 624.

In addition, the handling module 828 has a sealing element 804 which seals against the base module 622 in the operable and/or handleable state. The sealing element 104 is, for example, an O-ring. The sealing element 104 is preferably made of a, in particular elastic, deformable material, for example of rubber and/or an elastomer.

In addition, the handling module comprises an operating assembly 865, which is designed to correspond to the operating assembly 65. The base module 622 also comprises actuating elements corresponding to the base module 22. It should be noted that the operating assembly 865 in particular is optional and/or any number of operating elements may be provided.

The handling module 828 is a one-piece injection-molded part made of a plastics material that has particularly good heat-insulating properties. The user guiding the handling module 828 by hand is not affected during use, in particular by the heat-insulating effect of the handling module 828, although the base module 622, in particular the housing 624, can heat up during operation. In order to be able to keep the base module 622 in a suitable temperature range and/or to dissipate heat from the base module 622 to the environment, the air inlets 806 and the air outlets 810 are provided. Ambient air can enter the free space 860 through the air inlets 806, absorb heat and exit again through the air outlets 810. The air can therefore circulate around the housing 624 and cool it by convection. In particular, a suitable distance must be provided between the housing 624 and the base module 828 in order to achieve air circulation, in particular as per the arrows shown in FIG. 17.

In the illustration shown, the base module 622 comprises a securing element 840, which is arranged on a shaft 670 of the base module 622 in a close range to the housing 624, in particular immovably, and a supply cable 88, which is releasably connected to the base module 88. The supply cable 88 does not comprise a securing element. Referring to FIG. 12 and FIG. 14, it should be noted that the supply cable 88 is compatible with, or connectable to, the base modules shown there. If, for example, air cooling according to FIG. 17 or the handling module 828 is provided for handling the base module, the supply cable 88 can be connected to the base module without a securing element, in particular by the user.

The handling module 828 and the base module 622 together form a securing mechanism 834, as described below. In the operable state, the securing element 840 is arranged axially within the handling module 828. Furthermore, the securing element 840 is arranged distally in relation to the sealing element 804. The securing element 840 comprises two radial projections 871 extending from circumferentially opposite sides of the shaft 670. According to FIG. 18, the handling module 828 comprises a holding element 838 and two recesses 866 formed corresponding to the projections 871. The holding element 838 is configured to releasably secure the housing 624 to the handling module 828 by engaging behind it. The recesses 866 are formed such that the projections 871 can be guided through the recesses 866, in particular during the introduction of the base module 622 into the accommodation space 832. As soon as the projections 871 are arranged distal to an axial outer surface, or end surface, of the holding element 838, or completely guided through the recesses 866, the base module 622 together with the shaft 670 and the projections 871 can be rotated in order to bring the base module 622 into a securing position.

In order to guide the projections 871 completely through the recesses 866, the sealing element 804 is axially compressed. In particular, the housing 624, in particular in the introduced state and/or during introduction, presses on the sealing element 804. As a result, the sealing element 804 exerts an axial force on the housing 624 opposite to an introduction direction of the base module 622. In particular, the sealing element 804 presses on the housing 624 even after the base module 622 has been rotated. As a result, the securing element 840 presses on the holding element 838 from the distal side. In particular, the handling module can be clamped by the sealing element 804 and the projections 871 and/or is clamped in the operable state. In order to release the handling module, the housing can be pressed axially against the sealing element 804 and, as a result, the base module 622 can be rotated so that the projections 871 can be guided through the recesses 866.

To introduce the base module 622 into the accommodation space 832 and/or to secure the base module 622 to the handling module 828, in particular to establish the operable state, the user thus brings the shaft 670 out of the accommodation space 832 on the distal side of the handling module 828. Furthermore, the user guides the projections 871 through the recesses 866. In doing so, the user presses the housing 624 against the sealing element 804. Once the projections 871 are arranged distal to the retaining element 838, the user rotates the base module 622 to establish the operable state and consequently no longer exerts force on the base module 622 and/or the housing 624. In the operable state, the sealing element 804 presses the housing 624 in the axial direction such that the handling module 828 is clamped between the projections 871 and the housing 624. To release, the user presses the housing 624 against the sealing element 804 and rotates the base module 622 such that the projections 871 can be guided through the recesses 866 and guides the projections 871 through the recesses 866.

FIG. 20 shows a further embodiment of a handling module 928 in a schematic representation, wherein the base module 622 is shown in an introduced state, or ready-to-use state, in an accommodation space 932 of the handling module 928. The handling module 928 is very similar at least to the handling module 828. Therefore, the differences shall primarily be discussed. In particular, with regard to a securing mechanism 934, reference is made to the securing mechanism 834, which are designed in the same way. The present embodiment of the handling module 928 is described below with the aid of FIG. 21 and FIG. 22. FIG. 21 shows a schematic representation of the handling module 928 as viewed from a distal side of the handling module 928. FIG. 22 shows a schematic representation of the handling module 928 as viewed from a proximal side of the handling module 928.

The primary difference between the handling module 928 and the handling module 828 is that the handling module 928 does not have openings through a distal end surface 917. In particular, it should be noted that the handling module 928, like the handling module 828, can be used or is compatible with many base modules. The handling module 928 is shown together with the base module 622 merely as an example. In addition, the handling module 928 has similar advantages to the handling module 828 in terms of thermal insulation. In particular, the handling module 928 is largely manufactured from a plastics material and/or may be formed at least substantially in one piece. In particular, a main body 930 is formed in one piece.

A coolant communication interface 921 is provided for cooling the base module 622. In particular, the handling module 928 comprises the coolant communication interface 921, via which a cooling fluid, in particular cooling air, is introducible into the accommodation space 932. A tube 971 can be releasably fastened to the coolant communication interface 921. The tube 971 can be releasably fastened to the coolant communication interface 921, for example by means of a rotary coupling, in particular comprising a union nut, a claw coupling or a plug-in coupling. Furthermore, the tube 971 can be coupled, for example, to the supply unit 12 and/or an air supply unit which is configured to provide cooling air. In particular, the cooling air may be provided with a controllable and/or adjustable pressure and/or flow.

The cooling air can be directed and/or forced into a duct 969 via the tube 971. The duct 969 extends within a lamella 929 that extends from the proximal end of the handling module 982 to a distal section 908 of the handling module 928. In particular, the lamella 929 extends over 85% of the length of the housing 624. In other embodiments, the blade may extend, for example, over 90%, 95%, 80%, and/or any other distance relative to the housing 624. The lamella 929 is formed by the handling module 928 and protrudes from a radial inner surface of the handling module 928 into the accommodation space 932. In particular, at least one radially inner point of the lamella 929 contacts the base module 622 in the operable state. The lamella may have similar properties, in particular guiding properties and/or centering properties, to the support elements 869, 169 and/or the guide lamella 758. At the distal section 908, the duct 969 opens into the accommodation space 932 and defines an air inlet 906.

In addition, the handling module 928, similar to the handling module 828, has a sealing element 904 which seals against the base module 622 in the operable state. In addition, the sealing element 904 has the same properties with respect to the securing mechanism 934 as with respect to the securing mechanism 834. The accommodation space 934 is sealed from the environment at the distal section 908. Air introduced through the air inlet 906 into the accommodation space 932 introduced via the coolant communication interface 921. The air flows in the direction of the proximal section 912 as shown by the arrows in FIG. 20. In particular, the air flows along a hollow cylindrical air duct 914 defined by a free space 960, similar to the free space 860. A radially inner surface 916 of the air duct 914 is defined by the housing 624. A radially outer surface of the air duct 914 is defined by a radially inner surface of the handling module 928. At the proximal section 912, the handling module 928 comprises three air outlets 910. The air outlets 910 are, as already described, connected to the air inlet 906. Air flowing into the accommodation space 932, in particular the air duct 914, through the air inlet 906, flows out of the accommodation space 932, in particular the air duct 914, through the air outlets 910. The air can absorb and/or dissipate heat from the base module 622 as described in connection with the handling module 828. In contrast, the amount of air introduced into the accommodation space 932 through the air inlet 906 can be controlled and/or regulated as required and/or according to the specific application. For example, if a greater cooling effect is required, a larger volume of air is introducible and/or air at a lower temperature is introducible.

The air outlets 910 extend along the circumference of the handling module 928. This is particularly evident in FIG. 22. The air outlets 910 are interrupted on the circumference by the lamella 929, which encompasses the duct 969, and two further lamellae 929. The other lamellae 929 are provided with the same reference numeral for clarity. The lamellae 929 together form a ring, viewed from the proximal side, which tapers in the axial direction. In particular, the lamellae 929 together form a cone. A radially innermost section of the cone defines an inner diameter that at least substantially corresponds to an outer diameter of the housing 624. The lamella 929, which encompasses the duct 969, does not taper, unlike the other two lamellae 929. In the operable state, the lamellae 929 are configured to guide the base module 622 axially and to define a radial preferred position. In particular, the lamellae 929 are configured to center the base module 622. The lamellae 929 may have a similar function to support elements 169, 869 with regard to the introduction of a base module and/or spatial alignment of a base module. The further lamellae 929 extend over the same distance to the same axial position as the lamella 929 which encompasses the duct 969. On the distal section 908, the lamellae 929 in particular do not extend to the distal end surface 917, or to a wall defining the distal end surface 917. The air can flow around the lamellae 929 on the distal section 908.

A further embodiment of a handling module 1028 is shown in FIG. 23 and FIG. 24. FIG. 23 shows a schematic representation of a proximal section 1012 of a handling module 1028, wherein the base module 622 is shown in an introduced state, or ready-to-use state, in an accommodation space 1032 of the handling module 1028. The handling module 1028 is very similar at least to the handling module 928. Therefore, the differences shall primarily be discussed. FIG. 24 shows a schematic representation of the handling module 1028 as viewed from a proximal side of the handling module 1028.

In contrast to the handling module 928, the handling module 1028 is configured to enable liquid cooling of the base module 622. The handling module 1028 comprises, on the proximal section 1012, a support element 1069 which is designed as a ring element. The support element 1069 is formed integrally with the handling module and/or extends from a radial inner surface of the handling module 1028 into the accommodation space 1032. The handling module 1028, in particular the support element 1004, also has a sealing element 1004 which is designed as an O-ring. The sealing element 1004 is partially received by a sealing element circumferential groove on a radial inner surface of the support element 1069 and/or seals against the base module 622, in particular the housing 624, in the operable state. The support element 1069 also tapers conically. The support element 1069 can guide, align, center and/or support the base module 622 in accordance with the other support elements and/or the like.

The distal section of the handling module 1028 is designed similarly to the distal section 908 of the handling module 928. In particular, the accommodation space 1032 is sealed from the environment at the distal section. In general, in the handleable state, the accommodation space 1032 is sealed from the environment.

In addition, the handling module 1028 comprises a coolant communication interface 1021, via which a cooling fluid is introducible into the accommodation space 1032. The handling module 1028, in particular the coolant communication interface 1021, comprises two tubes 1071 which are guided through the support element 1069. In particular, the tubes 1071 are permanently connected to the handling module 1028. The handling module 1028 also as a single-use product and is disposed of and/or recycled together with the tubes 1071 after use. The cooling fluid, for example water and/or any other suitable liquid coolant, is introducible into the accommodation space 1032 via one of the tubes according to the arrows shown in FIG. 23 and FIG. 24. The tube 1071, by means of which the cooling fluid is introduced, extends to the distal section not shown. At the distal section, the cooling fluid is introduced into the accommodation space 1032. The tubes 1071 can in particular be coupled to the supply unit 12, a pump and/or a coolant supply unit. These may be configured to introduce the cooling fluid, similarly to the cooling air as per the handling module 928, into the accommodation space 1032 in a controllable and/or adjustable volume flow and/or pressure and/or with a controllable and/or adjustable temperature.

The handling module 1028 also comprises at least two lamellae 1029, in the present case four lamellae 1029, of which, however, only one is visible in the figures. The lamellae are designed similarly to the lamellae 929 and, in particular, contact the housing 624 along their longitudinal direction. The lamella 1029, which is shown in FIG. 23, extends to a distal end surface, or its accommodation space-side wall, in the distal section, not shown, of the handling module 1028. The cooling fluid introduced at the distal section is directed to the proximal section through a fluid duct. A radially inner surface of the fluid duct is defined by the housing 624 and a radially outer surface by a radially inner surface of the handling module. Side surfaces of the fluid duct are defined in particular by the lamellae 1029.

The illustrated lamella 1029 does not extend to the support element 1069 on the proximal section 1012. In particular, a proximal end 1073 of the lamella 1029 is spaced from the support element 1069. In particular, the lamella 1029, in particular the proximal end 1073 of the lamella 1029, is spaced from the support element 1069 such that the cooling fluid can pass the lamella 1029 circumferentially. A circumferentially adjacent lamella 1029 extends, in contrast, from the support element 1069 to a distal end, wherein the distal end is spaced from the distal end surface such that the cooling fluid can pass the lamella 1029 circumferentially at the distal section. Further lamellae 1029 may extend in the same pattern such that they are alternately spaced from the distal end surface and the support element. In summary, the cooling fluid can be guided along the housing in a labyrinth-like manner. The cooling fluid is led to the tube 1071 of the coolant communication interface 1021 and out of the accommodation space 1032 via the tube 1071.

Advantageously, a better cooling performance compared to air cooling can be achieved by using a liquid cooling fluid. In addition, a smaller diameter of the handling module 1028 may be provided.

FIG. 25 shows a further aspect of the endoscope 320, comprising the handling module 328 and the base module 322, in a schematic representation. FIG. 26 shows a schematic representation of the handling module 328 in a sectional view of the proximal side of the handling module 328. With reference to these two figures, an exemplary embodiment of a base module cooling system is described below. It should be noted that this form of base module cooling system may be combined with other embodiments of handling modules, including those shown herein. The endoscope 320 comprises a cooling module 122, which is provided for cooling the base module 322. The cooling module 122 can be arranged within the handle 344. In particular, the cooling module 122 comprises a latent heat accumulator 126. The latent heat accumulator 126 is arranged in a housing of the cooling module 122. The housing has good heat-conducting properties. The main body 330 of the handling module 328 defines a cooling module accommodation space 124 into which the cooling module 122 is introducible. Furthermore, a longitudinal axis of the handle 344 of the endoscope 320 is inclined with respect to a longitudinal axis of the accommodation space 334. In particular, the handle 344 is a pistol grip.

In particular, the user can introduce the cooling module into the cooling module accommodation space 124 and/or establish a handleable state. In an introduced state of the cooling module 122 in the handleable state, the cooling module 122 and the base module 322 are at least thermally in contact. This may mean that the cooling module 122 is in thermal contact with the base module 322 indirectly via, for example, heat conductors, in particular copper elements, or directly. In the form shown, the cooling module 122 is introducible from a lateral side into the handle 344, in particular into the cooling module accommodation space 124. In addition, the cooling module accommodation space 124 opens into the accommodation space 332 of the handling module 328. In the handleable state, the cooling module 122 can be brought into physical and thermal contact with the base module 322 by being introduced into the cooling module accommodation space 124. Furthermore, the base module 322 and/or the cooling module 122 has a heat-conducting deformable element 128 which, in the handleable state and in the introduced state, rests against the cooling module 122 or the base module 322. The heat-conducting deformable element has similar properties to the elastic heat-conducting element 352 and/or may be formed from a heat-conducting elastomer, for example. In the present case, the heat-conducting deformable element 128 is arranged on the cooling module 122. Waste heat is dissipated via the heat-conducting element 128 and/or conducted from the base module 322 to the cooling module 122. In addition, the heat-conducting element 128 can clamp the base module 322, whereby it is securely held in the accommodation space 332. In particular, in the handleable state and in the introduced state of the cooling module 122, the cooling module 122 applies a force to the base module 322. A fastening mechanism 321 is provided to maintain the application of force. The fastening mechanism 321 comprises two push buttons 323, two locking lugs 325 and a locking hole 327. The push buttons 323 are arranged laterally on the main body 344. In addition, the push buttons are mechanically connected to the locking lugs via a lever arrangement. The locking lugs 325 partially protrude into the cooling module accommodation space 124. By operating the push buttons 323, the locking lugs 325 can be moved at least substantially out of the cooling module accommodation space 124. Furthermore, the locking lugs 325 can be moved out of the cooling module accommodation space 124 by introducing the cooling module 122 into the cooling module accommodation space 124. In the introduced state of the cooling module 122, the locking lugs 325 engage in the locking hole 327. In particular, the locking lugs 325 hold the cooling module 122 by engaging behind it in the cooling module accommodation space 124. Furthermore, the user can apply a force to the heat-conducting deformable element 128 during the introduction of the cooling module 122. The locking lugs 325 are configured to hold the cooling module 122 in the cooling module accommodation space 124 in such a way that the base module 322, in particular the heat-conducting elastic element 128, is subjected to a force. To remove the cooling module 122, the user can operate the push buttons 323. In this case, the force applied to the base module 322 causes the cooling module 122 to move against the direction in which the cooling module 122 is introduced.

FIG. 27, FIG. 28 and FIG. 29 show a schematic detailed representation of the operating assembly 65 and the associated operating elements 64. FIG. 27 shows a schematic side view of a cutout of the base module 22, a cutout of the main body 30 of the handling module 28 and the operating assembly 65. The operating assembly 65 has already been outlined in connection with FIG. 4. It can be seen in FIG. 27 that the operating assembly 65 can be manufactured separately from the main body 30. The operating assembly 65 comprises a housing 69 and three operating elements 64. Two of the operating elements 64 are designed as push buttons 67. The third operating element 64 is designed as a rotary knob 70.

The actuating elements 64, in particular the push buttons 67, each comprise a magnet 66, which is in particular a rare earth magnet, for example comprising neodymium, dysprosium and/or samarium. In the embodiment shown, the magnet 66 of the push buttons 67 comprises two poles arranged consecutively in the radial direction. Furthermore, the push buttons 67 are guided in such a way that they can be moved in the radial direction. By actuating, in particular pressing, the push buttons 67, the respective magnet 64 can be moved in the direction of an associated actuating element 62 of the base module 22.

Furthermore, the rotary knob 70 comprises a magnet 66, which is in particular a rare earth magnet, for example comprising neodymium, dysprosium and/or samarium. This magnet 66 comprises two poles arranged consecutively in the axial direction. By actuating, in particular pressing, the push buttons 67, the respective magnet 64 can be moved in the direction of an associated actuating element 62 of the base module 22. By actuating, in particular rotating, the rotary knob 70, the magnet 64 can be moved relative to an associated actuating element 62 of the base module 22, in particular rotated, or moved to a different rotational position.

In principle, instead of at least one magnet 66, a component made of a ferro-, ferri-and/or paramagnetic and/or other electrically conductive material may be provided.

At least one of the actuating elements 62 may comprise a Hall sensor 68. In the present example, all actuating elements 62 each comprise a Hall sensor 68. The function of the actuating elements 62 is explained by way of example using an actuating element. By means of the actuating element 62 comprising the Hall sensor 68, a, in particular local, magnetic flux density of the magnet 66 of a push button 67 can be measured. By knowing the magnetic flux density, a relative radial position of the push button 67, and/or of the magnet 66 associated with the push button 67, to the actuating element 62 can be deduced. The push button 67, together with the actuating element 62, can form a switch that can be switched between two positions. The switch may in particular be a proximity switch, for example a magnetic proximity switch, a capacitive proximity switch, an optical proximity switch and/or an inductive proximity switch. Furthermore, the switch can be switchable between more than two positions, for example three, four or five positions. The positions are defined by a radial relative position of the push button 67, in particular of the corresponding magnet 66, to the actuating element 62, in particular the corresponding Hall sensor 68. The push button 67 may comprise a return element configured to return the push button 67 to an initial position after actuation. The return element may be an elastic element, in particular a spring element.

Furthermore, a rotational position of the rotary knob 70 and/or the magnet 66 associated with the rotary knob relative to the actuating element 62 may be closable. The actuating element 62 may comprise multiple, in particular two, three or four Hall elements 62, in particular for measuring the rotational position. The actuating element 62 may be configured to measure the rotational position with a resolution of, for example, 0.1° to 20°, in particular 0.5° to 10° and preferably 1.0° to 5°. The associated actuating element 62 is actuated in accordance with the rotational position.

FIG. 28 shows a schematic view of the operating assembly 65 from the proximal side. In particular, the rotary knob 70 comprising the magnet 66 and the actuating element 62 associated with the rotary knob 70, which comprises at least one Hall sensor 68, can be seen. Furthermore, a further aspect that has already been described in connection with FIG. 3 can be seen. In FIG. 28, in addition to the actuating element 62 associated with the push button 70, a further actuating element 62, which is of identical construction and/or functionally identical to the other actuating element 62, can be seen. The further actuating element 62 is opposite the other actuating element 62. By rotating the base module 22, which may be advantageous in particular with regard to a viewing direction of the endoscope 20, the further actuating element 62 can be arranged with respect to the operating element 64, in particular the rotary knob 70, in such a way that it can interact with the latter.

FIG. 29 is a schematic plan view of the operating assembly 65. The three actuating elements 64 can be seen, of which two are designed as push buttons 67 and one as a rotary knob 70. Also visible is the housing 69 of the operating assembly 65, into which the actuating elements 64 are at least partially embedded. In embodiments not shown, the operating assembly comprises one, two, four, five, six and/or seven actuating elements 64.

FIG. 30 and FIG. 31 show a further aspect of the supply cable 88; the embodiments shown differ only slightly. Referring to FIG. 30, it can be seen that the supply cable 88 comprises a plug 91 of the plug connector 90 already described in connection with FIG. 3. The plug 91 extends along the longitudinal axis 96 of the base module (not shown). The supply cable 88 is rotatable when connected to the base module. Furthermore, it can be seen that a cable outlet direction 94 of the supply cable 88 from the base module is inclined with respect to the longitudinal axis 96 of the base module. In the case shown, the cable outlet direction 94 is inclined by 90° relative to the longitudinal axis 96. The inclination of the cable outlet direction 94 is defined in particular by a transition section 92 of the supply cable 88 which connects to the plug 91. In the present case, the transition section 92 is made of hard plastics material. Alternatively or additionally, the transition section 92 may be formed from rubber, a plastics material and/or a metal. In particular, the supply cable 88 is reinforced in an area of the transition section 92.

FIG. 31 shows a section of a substantially similar supply cable 88. In contrast, the cable outlet direction 94 is inclined by 45° relative to the longitudinal axis 96.

FIG. 32 is a schematic view of a medical robot 132. The robot 132 comprises a robot arm 133, a robot control unit 129 and a holder 130. The holder 130 is arranged at a proximal end of the robot arm 133. The handling module 28 is configured to be attached to the holder 130 of the medical robot 132 and to make the base module 22 handleable by the robot 132. The holder 130 is formed together with the handling module 28. In other embodiments, the holder is formed separately from the handling module. The robot arm 133 enables the base module 22 to be moved in all spatial directions and/or pivoted about at least one, in particular adjustable, spatial point.

FIG. 33 shows a schematic flow chart of a method for providing the endoscope 20. For a detailed description, reference is made to the explanations at least in connection with FIGS. 1 to 4. The method comprises at least a step 1100 of providing the base module 22, a step 1102 of providing the handling module 28 and a step 1104 of introducing the base module 22 into the handling module 28 by the user. It is understood that the described method is transferable to at least all embodiments of base modules and handling modules according to the invention and/or shown.

In other words, the following aspects are described here, among others.

A base module may comprise an endoscope shaft and a proximal end piece with an interface to the CCU (computer control unit). The base module may also comprise sensors for detecting key magnets. Functions for generating an image (camera and illumination) may be located within a hermetically sealed housing, which ensures autoclavability. A special feature in this respect may be the installation of a magnetic sensor on two opposite areas of the housing. This is advantageous for angled-view endoscopes, as the user can determine whether the viewing direction is upwards or downwards in relation to the buttons by sliding the endoscope into the handle at an angle of 180°.

A cable interface is preferably coaxial. This allows the rotation of a non-0° cable outlet in the direction required in the respective application. The user can also change the cable in the event of a defect, as no rotation angle on the axis needs to be taken into account during the plugging process. Owing to the serializer in the base module and an additional filter for power transmission, a simple coaxial cable is sufficient to connect to the CCU. These simple cables are so inexpensive to procure that a single use could also be considered. If autoclavable materials are used for the cable, the cable can remain plugged in at all times.

This solution is advantageous because the user does not have to make any further electrical connections
in addition to the connection to the CCU,
which is always necessary.

In hand-held applications, a handle part takes over the tasks of endoscope guidance, thermal insulation and control of the electrical functions (buttons, rotary knob).

In some variants, the base module can be slid into the handle from proximal to distal and locked with the cable plugged in.

If a base module is located centrally within the handling module, it can transfer its power loss directly to the air flowing through it. By dispensing with any in the handling module, the latter can be designed as a disposable plastics material part (the cost of any magnets is negligible). The poor thermal conductivity of the plastics material insulates the user from high temperatures on the base module surface. This has a positive effect on heat dissipation, as the higher the temperature difference between the incoming cooling air and the base module surface, the better the convection. If the LEDs are installed proximally, they can be operated at a higher power due to better heat dissipation, which means that a reserve is available in the illumination for critical situations such as heavy bleeding or the operating site can be better illuminated overall.

If a cooling fluid is introduced into the space between the base module and the handle via a supply tube, the cooling fluid exits in a heated state at the proximal side. As the cooling fluid does not come into contact with the patient, the room air can be used for this purpose. The overall diameter of the handle can be reduced, resulting in a handling advantage.

If liquid cooling is used, a return line and a seal may be provided in the proximal area. At the same volume flow, a significantly larger amount of heat can be dissipated than with air due to the high heat capacity of water or other cooling liquids. This variant is therefore suitable for high-power LEDs installed proximally in the base module, which make a stationary light source obsolete for most applications.

When using a media cooling system, the single-use design of the handling module is preferable, as the necessary supply and return tubes can be integrated into the handle part and no problems arise during reprocessing.

For positioning within the handling module, there may be centering bevels in the front part which, together with the rear guide and the spring force of the locking mechanism, ensure a precise and fixed position. Different embodiments may be identical or analogous to one another, in particular except for one type of seal. There may be a snap-in spring in the rear part that automatically locks when it is slid in. To unlock and pull out the base module, the spring must be moved into an unlocking position by applying manual pressure. The situation is similar with a locking option where the tension of the spring can be generated by turning it in on a slightly inclined path. The end position can be slightly recessed in relation to the bevel so that the spring engages and can only be unlocked again by compressing and rotating. The options shown are possible solutions in principle, which are primarily intended to demonstrate the ease of handling during assembly in the operating theater preparation.

The buttons on pistol grip variants are operated using a simple mechanism that moves the magnets to the correct position on the base module and thus triggers the desired function. With a pistol grip, the cable outlet is preferably at the knob at the bottom. For this purpose, the base module can be equipped with a cable variant with an oblique angle to the plug-in shaft.

Another grip variant is a pen-like grip, as explained. The endoscope is in this case constructed in the manner of a so-called penscope. In this case, the pushed-on part is extended forwards. However, the diameter in the extended part is only slightly larger than the shaft diameter, so that the instrument can be held and rotated like a pencil between the thumb and index finger. The larger diameter lies on the metacarpus in the area between the thumb and index finger. This variant is preferred in particular in neurosurgery.

In some variants, the power loss generated in the base module can be transferred to a cooling module filled with phase change material. Due to the phase transition, a large amount of energy can be stored in the cooling module using a small volume. This makes it possible, for example, to dispense with water cooling and the additional tubes, a water reservoir and a pump that this requires. The system can be operated indefinitely because the cooling module can be easily replaced. For better heat transfer, the cooling module may be equipped with an elastic but highly heat-conducting material at the contact surface with the base module, thus ensuring a large or maximized heat exchange surface area.

Particularly in variants with a pistol grip, it may be provided that this clamps the base module after the clip closure has been closed. The pistol grip can then absorb the power loss via an elastic heat-conducting material and distribute it over a larger surface. Due to the elastic material, the base module may no longer be slid in in some embodiments, but is conveniently clamped. The principle is not limited to a pistol grip. Depending on the power to be dissipated, simply increasing the diameter of a round handle may be sufficient to keep temperatures within the user's comfort zone.

Various user input elements are possible on the basis of contactless magnetic sensors. In addition to touch functions, an analog input can also be made via a rotation angle sensor. This type of input is advantageous for controlling image focus or a continuous electronic zoom. In particular, a rotation angle sensor is very sensitive regarding the correct distance on the Z-axis. The round base housing can be flattened in the area of this sensor so that the magnet can be positioned as close as possible to the sensor. Likewise, the material of the base module housing can be diamagnetic, which is the case for steel, for example.

If the selectability of the button position relative to the oblique viewing direction is desired and the Hall sensor system is installed mirrored, the option of making inputs on the top and bottom can be used to place only sparingly used functions or functions that should not be unintentionally adjusted during operation (for example, focusing with a rotary knob) on the side that is more difficult to access.

In principle, the slide-in direction of the base module can also be reversed, i.e., it is slid in from distal to proximal. This is advantageous if the accommodating handling module is equipped with electronics and is not autoclaved, but is covered with a drape for each application.

### List of Reference Signs

10 system
12 supply unit
14 display unit
16 anatomical structure
20 endoscope
22 base module
24 housing
26 control electronics
28 handling module
29 handling module
30 main body
32 accommodation space
34 securing mechanism
36 pretensioning element
38 holding element
42 main body parts
44 handle
62 actuating element
64 operating element
65 operating assembly
66 magnet
67 push button
68 Hall sensor
69 housing
70 rotary knob
70 shaft
71 interface
72 distal section
74 proximal section
78 camera
80 light source
82 optical element
84 main viewing direction
88 supply cable
90 plug connector
91 plug
92 transition section
94 cable outlet direction
96 longitudinal axis
98 proximal side
100 opening
104 sealing element
122 cooling module
124 cooling module accommodation space
126 latent heat accumulator
128 heat-conducting deformable element
129 robot control unit
130 holder
132 medical robot
133 robot arm
134 sterile single-use product
136 sterile outer packaging
148 image sensor
150 electronic elements
152 heat pipe
154 distal opening
156 distal side
158 actuating mechanism
160 free space
162 distal section
163 distal section
164 additional alignment aid
166 cable base
169 support element
220 endoscope
222 base module
224 housing
228 handling module
230 main body
232 accommodation space
244 handle
252 diameter
254 diameter
320 endoscope
321 securing mechanism
322 base module
323 push button
325 locking lug
327 locking hole
328 handling module
332 accommodation space
334 securing mechanism
336 locking element
338 locking receptacle
340 main body part
342 main body part
344 handle
346 longitudinal axis
348 longitudinal axis
350 hinge
352 heat-conducting element
362 actuating element
364 operating element
366 magnet
368 Hall sensor
404 sealing element
422 base module
428 handling module
432 accommodation space
470 shaft
471 interface
522 base module
528 handling module
534 securing mechanism
540 securing element
570 shaft
622 base module
624 housing
628 handling module
632 accommodation space
634 securing mechanism
636 pretensioning element
638 holding element
640 securing element
643 guideway
645 locking section
670 shaft
688 supply cable
696 longitudinal axis
722 base module
724 housing
728 handling module
732 accommodation space
758 guide lamella
761 guide groove
760 free space
804 sealing element
806 air inlet
808 distal section
810 air outlet
812 proximal section
814 air duct
816 radial inner surface
817 distal end surface
828 handling module
832 accommodation space
834 securing mechanism
838 holding element
840 securing element
860 free space
865 operating assembly
866 recess
867 web
869 support element
871 projection
904 sealing element
906 air inlet
908 distal section
910 air outlet
912 proximal section
914 air duct
916 radial inner surface
917 distal end surface
921 coolant communication interface
928 handling module
929 lamella
930 main body
932 accommodation space
934 securing mechanism
958 guide lamella
960 free space
969 duct
971 hose
1004 sealing element
1012 proximal section
1021 coolant communication interface
1028 handling module
1029 lamella
1032 accommodation space
1060 free space
1069 support element
1071 hose
1073 proximal end
1100 step
1102 step
1104 step

1. Endoscope (20, 220, 320), comprising:
a base module (22, 622), comprising:
a housing (24, 624); and
control electronics (26, 626), wherein the housing (24, 624) encloses the control electronics (26, 626); and
a handling module (28, 628, 828, 928, 1028) configured to make the base module (22, 622) handleable and comprising a main body (30, 630, 830, 930, 1030) that defines an accommodation space (32, 632, 832, 932, 1032) for the base module (22, 622);
wherein, to establish a handleable state, the base module (22, 622) is introducible into the accommodation space (32, 632, 832, 932, 1032) of the handling module (28, 628, 828, 928, 1028) by a user, and
wherein the handling module (28, 628, 828, 928, 1028) and the base module (22, 622) together form a securing mechanism (34, 634, 834, 934, 1034) by means of which the housing (24, 624) is releasably securable to the handling module (28, 628, 828, 928, 1028).

2. Endoscope (20) according to claim 1,
wherein the handling module (28, 628, 828, 928, 1028) is configured to make the base module (22, 622) handleable and operable by the user; and
wherein an operable state can be established by introducing the base module (22, 622) into the accommodation space (32, 632, 832, 932, 1032).

3. Endoscope (20) according to claim 1 or 2,
wherein the base module (22, 622) is releasable from and/or securable to the handling module (28, 628, 828, 928, 1028) without tools.

4. Endoscope (20) according to any one of the preceding claims, wherein the securing mechanism (34, 634) comprises a pretensioned and/or pretensionable pretensioning element (36, 636).

5. Endoscope (20) according to any one of the preceding claims, wherein the securing mechanism (34, 634, 834, 934, 1034) comprises a holding element (38, 638, 838, 938, 1038) which is configured to releasably secure the housing (24, 624) to the handling module (28, 628, 828, 928, 1028) by engaging behind it.

6. Endoscope (20) according to any one of the preceding claims,
whereby the securing mechanism (634) has at least one rotatable securing element

(640) wherein the rotatable securing element (640) is movable by rotation between a securing state and a release state.

7. Endoscope (320) according to any one of the preceding claims,
wherein the accommodation space (332) is openable and closable.

8. Endoscope (320) according to claim 7,
wherein in an open state of the accommodation space (332) the base module (322) is introducible into the accommodation space (332), and
wherein in a closed state of the accommodation space (332) the housing (324) is secured to the handling module (328).

9. Endoscope (320) according to claim 7 or 8, wherein the main body (330) comprises at least two main body parts (340, 342) which are connected to one another in an articulated manner and are movable relative to one another for opening and closing the accommodation space (332).

10. Endoscope (20) according to any one of the preceding claims,
wherein the main body (330) comprises a handle (44).

11. Endoscope (20) according to claim 10, wherein in the handleable state the handle (44) encompasses the housing (24) at least in sections.

12. Endoscope (20) according to claim 10 or 11, wherein in the handleable state the handle (44) is arranged, at least in sections, distally with respect to the housing (24).

13. Endoscope (20) according to claim 10 or 11, wherein in the handleable state the handle (44) is arranged, at least in sections, proximally with respect to the housing (24).

14. Endoscope (20) according to any one of claims 10 to 13,
wherein a longitudinal axis of the handle (46) corresponds to a longitudinal axis of the accommodation space (32).

15. Endoscope (320) according to any one of claims 10 to 14,
wherein a longitudinal axis of the handle (344) is inclined with respect to a longitudinal axis of the accommodation space (334).

16. Endoscope (320) according to claim 15,
wherein the handle (344) is a pistol grip.

17. Endoscope (220) according to any one of claims 10 to 16,
wherein the handle (244) has a smaller diameter (252) than the housing (24).

18. Endoscope (20) according to any one of claims 10 to 17,
wherein the handle (44) has an ergonomic shape.

19. Endoscope (20) according to any one of the preceding claims,
wherein the handling module (28, 728) and the base module (22, 722) together form an alignment aid (56, 756) which defines a preferred position and/or a preferred orientation of the housing (24, 724) relative to the handling module (28, 728).

20. Endoscope (20) according to claim 19,
wherein the alignment aid (56, 756) has at least one guide lamella (758) and a guide groove (761) corresponding to the guide lamella (758).

21. Endoscope (20) according to any one of the preceding claims,
wherein the base module (22) comprises at least one actuating element (62) by means of which at least one function of the base module (22) can be controlled; and
wherein the handling module (28) comprises at least one operating element (64) which, in the handleable state, acts on the actuating element (62) in such a way that an operation of the operating element (64), in particular by the user, causes an actuation of the actuating element (62).

22. Endoscope (20) according to claim 21, wherein the operating element is rotatable, and
wherein an actuation of the actuating element (62) takes place in accordance with a rotational position of the operating element (64).

23. Endoscope (20) according to claim 22,
wherein the actuating element (62) and/or the operating element (64) comprises at least one magnet (66).

24. Endoscope (20) according to claim 23, wherein the rotary bearing comprises a slide ring (68).

25. Endoscope (20) according to claim 23 or claim 24,
wherein the base module (22) comprises at least two functionally identical actuating elements (62) which are arranged at different circumferential positions, in particular opposite one another with respect to a longitudinal axis of the accommodation space (32), and which are each configured to interact with the operating element (64).

26. Endoscope (20) according to any one of the preceding claims,
wherein the base module (22) further comprises a shaft (70) having a distal section (72) and having a proximal section (74), wherein the proximal section (74) is attached to the housing (24).

27. Endoscope (20) according to claim 26,
wherein the shaft (70) is designed as an interchangeable shaft.

28. Endoscope (20) according to claim 26 or 27,
wherein the distal section (72) comprises a camera (78) and/or a light source (80).

29. Endoscope (20) according to any one of claims 26 to 28,
wherein the distal section (72) comprises at least one optical element (82) defining a main viewing direction (84), wherein the main viewing direction (84) is inclined with respect to a longitudinal axis of the shaft (86).

30. Endoscope (20) according to any one of the preceding claims,
further comprising a supply cable (88) which is connected and/or connectable to the base module (22).

31. Endoscope (20) according to claim 30, wherein the supply cable (88) is permanently connected to the base module (22).

32. Endoscope (20) according to claim 30 31,
wherein the base module (22) and the supply cable (88) together form a plug connector (90).

33. Endoscope (20) according to any one of claims 30 to 32, wherein the supply cable (88) is a coaxial cable (92).

34. Endoscope (20) according to any one of claims 30 to 33, wherein the supply cable (88) is rotatable relative to the base module (22) in a connected state.

35. Endoscope (20) according to any one of claims 30 to 34, wherein a cable outlet direction (94) of the supply cable (88) from the base module (22) is inclined with respect to a longitudinal axis of the base module (96).

36. Endoscope (20) according to any one of the preceding claims,
wherein the main body (30) forms on its proximal side (98) an opening (100) through which the base module (22) is introducible into the accommodation space (32).

37. Endoscope (20) according to any one of the preceding claims,
wherein the control electronics (26) are enclosed in a gas-tight manner.

38. Endoscope (20) according to any one of the preceding claims,
wherein the handling module (28) is configured to be autoclaved and/or sterilized separately from the base module (22).

39. Endoscope (20) according to any one of the preceding claims,
wherein the handling module (28) is made at least partially and in particular at least largely from plastics material.

40. Endoscope (20) according to any one of the preceding claims,
wherein the main body (30) is manufactured by injection molding and/or additively.

41. Endoscope (20) according to any one of the preceding claims,
wherein the handling module (28) is a single-use product.

42. Endoscope (20) according to any one of the preceding claims,
wherein in the operable state a longitudinal axis of the base module (96) coincides with a longitudinal axis of the accommodation space (32).

43. Endoscope (20) according to any one of the preceding claims,
wherein the handling module (28, 828, 928) has at least one sealing element (104, 804, 904, 1004) which seals against the base module (22, 622) in the operable state.

44. Endoscope (20) according to any one of the preceding claims,
wherein the handling module (828, 928) further comprises at least one air inlet (806,

906) on a distal section (808, 908) of the handling module (828, 928) and at least one air outlet (810, 910) on a proximal section (812, 912) of the handling module (828, 928), wherein the at least one air inlet (806, 906) is connected to the at least one air outlet (810, 910).

45. Endoscope (20) according to claim 44,
wherein the at least one air inlet (806, 906) is connected to the at least one air outlet (810, 910) via an air duct (814, 914), wherein in the handleable state a radial inner surface (816, 916) of the air duct (814, 914) is defined by the housing (24, 624).

46. Endoscope (20) according to claim 45,
wherein the air duct (814, 914) is hollow cylindrical.

47. Endoscope (20) according to any one of the preceding claims,
wherein the handling module (28, 928, 1028) comprises a coolant communication interface (921, 1021) via which a cooling fluid is introducible into the accommodation space (932, 1032).

48. Endoscope (20) according to any one of the preceding claims,
wherein in the handleable state the accommodation space (32, 1032) is sealed from the environment.

49. Endoscope (320) according to any one of the preceding claims,
further comprising a cooling module (122),
wherein the main body (330) defines a cooling module accommodation space (124) into which the cooling module (122) is introducible, and wherein in an introduced state of the cooling module (122) in the handleable state, the cooling module (122) and the base module (322) are at least thermally in contact.

50. Endoscope (320) according to claim 49,
wherein the cooling module (122) comprises a latent heat accumulator (126).

51. Endoscope (320) according to claim 49 or 50,
wherein the cooling module accommodation space (124) opens into the accommodation space (332), and wherein in the handleable state the cooling module (122) can be brought into physical and thermal contact with the base module (322) by being introduced into the cooling module accommodation space (124).

52. Endoscope (320) according to any one of claims 49 to 51,
wherein the base module (322) and/or the cooling module (122) has a heat-conducting deformable element (128) which, in the handleable state and in the introduced state, rests against the cooling module (122) or the base module (322).

53. Endoscope (320) according to any one of claims 49 to 52,
wherein in the handleable state and in the introduced state of the cooling module (122), the cooling module (122) applies a force to the base module (322).

54. Endoscope (20) according to any one of the preceding claims,
wherein the handling module (28) is configured to be attached to a holder (130) of a medical robot (132) and to make the base module (22) handleable by the robot (132).

55. Base module (22) for an endoscope (20) according to any one of the preceding claims.

56. Handling module (28) for an endoscope (20) according to any one of the preceding claims.

57. Sterile single-use product (134), comprising:
a handling module (28) according to claim 56; and
a sterile outer packaging (136),
wherein the handling module (28) is packaged in a sterile state in the outer packaging (136).

58. System (10) comprising:
a base module (22) according to claim 55; and
at least two different handling modules (28), each according to claim 56, wherein the two handling modules (28) implement different functionalities in a handleable state of the base module (22).

59. Method for providing an endoscope (20) according to any one of claims 1 to 54 for a user, comprising:
providing the base module (22);
providing the handling module (28); and
introducing the base module (22) into the handling module (28) by the user.
